(19) United States
(12) Patent Application Publication
(10) Pub. No.: US 2025/0084469 A1
(43) Pub. Date: Mar. 13, 2025

(54) METHODS FOR ANALYZING NUCLEIC ACIDS USING SEQUENCE READ FAMILY SIZE DISTRIBUTION

(71) Applicant: GUARDANT HEALTH, INC., Palo Alto, CA (US)

(72) Inventors: Darya CHUDOVA, Los Altos, CA (US); Tingting JIANG, San Diego, CA (US); Catalin BARBACIORU, Fremont, CA (US); Marcin Pawel SIKORA, Palo Alto, CA (US)

(21) Appl. No.: 18/883,708

(22) Filed: Sep. 12, 2024

Related U.S. Application Data
(60) Provisional application No. 63/582,113, filed on Sep. 12, 2023.

(51) Int. Cl. C12Q 1/6851; C12Q 1/6855; G16B 20/10; G16B 20/20; G16B 30/10

(52) U.S. Cl. CPC C12Q 1/6851; C12Q 1/6855; G16B 20/10; G16B 20/20; G16B 30/10

(57) ABSTRACT

The present invention provides a method for determining a quantitative measure indicative of the number of nucleic acids in a sample that map to a specific genomic region. The method involves: (a) providing a sample containing parent nucleic acids; (b) amplifying these parent nucleic acids to generate progeny nucleic acids; (c) sequencing the progeny nucleic acids to produce sequence reads; (d) grouping the sequence reads into families, where each family corresponds to sequence reads derived from the same parent nucleic acid; and (e) utilizing both the number of families mapping to the genomic region and the family size distribution of these families to calculate a quantitative measure indicative of the number of nucleic acids in the sample that map to the genomic region. This method enhances the accuracy of quantifying nucleic acids within a genomic region, particularly in complex or low-abundance samples.

### CROSS-REFERENCE TO RELATED APPLICATIONS

This application claims priority to U.S. Provisional Application Ser. No. 63/582,113, filed on Sep. 12, 2023, the contents of which are herein incorporated by reference.

### BACKGROUND

Accurate quantification of nucleic acids in a biological sample is critical for a wide range of applications in genomics, including gene expression analysis, mutation detection, and genomic mapping. Traditional methods for quantifying nucleic acids, such as quantitative PCR (qPCR), often face limitations in sensitivity, accuracy, and the ability to distinguish between closely related sequences. These challenges are particularly pronounced when dealing with samples containing low-abundance nucleic acids or complex mixtures of closely related sequences.

Recent advances in high-throughput sequencing technologies have significantly improved our ability to sequence large quantities of nucleic acids with high precision. However, the accurate quantification of specific nucleic acid sequences within a sample, especially at low abundance, remains a significant challenge due to biases introduced during sample preparation, amplification, and sequencing processes. Amplification biases, in particular, can lead to over- or under-representation of certain sequences, resulting in inaccurate quantification.

### SUMMARY

The present invention addresses the need for an improved method to accurately quantify nucleic acids in a sample that map to a specific genomic region.

In an aspect, the present disclosure provides a method that allows for the determination of a quantitative measure indicative of a number of nucleic acids in a sample that map to a genomic region. This is achieved through the grouping of sequence reads from a nucleic acid sample into families, wherein family correspond to sequence reads of progeny nucleic acids derived from the same parent nucleic acid. The identified families can be used to determine the quantitative measure through the number of families that map to the genomic region and the family size distribution of families that map to the genomic region.

In some embodiments, a family of progeny nucleic acids can be identified by attaching molecular barcodes to the parent nucleic prior to amplification. Progeny nucleic acids from the same parent nucleic acid will contain the parent's molecular barcode, thus allowing the grouping of sequence reads of the progeny nucleic acids into families. Alignment of the sequence reads of the progeny nucleic acids can also contribute to the grouping of the sequence reads of the progeny nucleic acids into families according to the length of the sequence reads as well as the start and/or stop positions of the sequence reads when aligned to a reference sequence. The quantitative measure can then be determined through fitting the family size distribution to a statistical model.

In one aspect, the quantitative measure can be utilized in methods related to molecular counting. The number of families that map to the genomic region and the family size distribution of families that map to the genomic region can be used to infer information about ‘unseen’ molecules in that genomic region. For example, the family size distribution can be used to infer the number of nucleic acids in a sample that map to a genomic region which did not provide any sequence reads.

In another aspect, the quantitative measure can be used to provide information about the sample itself. For example, the quantitative measure can be used to detect copy number variation (CNV) in a sample. The quantitative measure could also provide information about hypermethylation and/or hypomethylation within a sample of nucleic acids. In another aspect, the family size distribution could be used to determine whether the genomic region is subject to experimental bias introduced by genomic factors. Identified bias can then be applied to other genomic regions to predict the experimental bias associated with further genomic regions.

Accordingly, in the first aspect, the present disclosure provides a method for determining a quantitative measure indicative of a number of nucleic acids in a sample that map to a genomic region, comprising: (a) providing the sample of parent nucleic acids; (b) amplifying the parent nucleic acids to provide progeny nucleic acids; (c) sequencing the progeny nucleic acids to provide sequence reads; (d) grouping the sequence reads into families, wherein a family corresponds to sequence reads derived from the same parent nucleic acid; and (e) using: (i) the number of families that map to the genomic region; and (ii) the family size distribution of families that map to the genomic region, to determine the quantitative measure indicative of the number of nucleic acids in the sample that map to the genomic region.

In some embodiments, the method further comprises aligning the sequence reads to a reference sequence.

In some embodiments, the parent nucleic acids are DNA. In some embodiments, the parent nucleic acids are cell free DNA. In some embodiments, the parent nucleic acids are complementary DNA (cDNA).

In some embodiments, step (e) comprises comparing the family size distribution to a reference value. In some embodiments, the reference value is: (i) a family size distribution of nucleic acids from the sample which map to one or more second genomic regions; or (ii) a mean family size distribution of sequence reads in families from the sample.

In some embodiments, step (e) comprises inferring from the family size distribution the number of parent nucleic acids in the sample that map to the genomic region which did not provide any sequence reads.

In some embodiments, the method further comprises detecting copy number variation in the sample by determining a normalized quantitative measure determined in step (e) at one or more genomic regions and determining copy number variation based on the normalized quantitative measure.

In some embodiments, the sample of parent nucleic acids has been subjected to a methylation-based partitioning assay. In some embodiments, the methylation-based partitioning assay partitions nucleic acids using methyl-binding domain (MBD). In some embodiments, the method is performed on (i) a hypermethylated partition obtained from the methylation-based partitioning assay and/or (ii) a hypomethylated partition obtained from the methylation-based partitioning assay. In some embodiments, thee method further comprises detecting a quantitative measure indicative of a number of nucleic acids in the hypermethylated and/or hypomethylated partition derived from a genomic region in the sample by determining a normalized quantitative measure determined in step (e) at one or more genomic regions and determining a methylation level at that genomic region based on the normalized quantitative measure.

In some embodiments, the grouping of the sequence reads into families is based at least in part on molecular barcodes. In some embodiments, the molecular barcodes are attached to the parent nucleic acids through: (i) ligation of adapters comprising the molecular barcodes; or (ii) amplification using primers comprising the molecular barcodes.

In some embodiments, the grouping of the sequence reads into families is based at least in part on the length of the sequence reads and/or the start and/or stop position of the sequence reads when aligned to a reference sequence.

In some embodiments, the quantitative measure indicative of the number of nucleic acids in the sample that map to the genomic region is determined by fitting the family size distribution to a statistical model. In some embodiments, the statistical model is a Poisson distribution or a negative binomial distribution.

In another aspect, the present disclosure provides a method of identifying whether a genomic region is subject to experimental bias, wherein the method comprises: (a) providing a sample of parent nucleic acids; (b) amplifying the parent nucleic acids to provide progeny nucleic acids; (c) sequencing the progeny nucleic acids to provide sequence reads; (d) grouping the sequence reads into families, wherein a family corresponds to sequence reads derived from the same parent nucleic acid; and (e) using the family size distribution of families that map to the genomic region to determine whether the genomic region is subject to experimental bias.

In some embodiments, step (e) comprises comparing the family size distribution to a reference value, wherein: (i) an increase in family size relative to a reference value represents a bias from over representation of nucleic acids in the sample that map to the genomic region; and (ii) a decrease in family size relative to a reference value represents a bias from under representation of nucleic acids in the sample that map to the genomic region.

In some embodiments, the reference value is: (i) a family size distribution of nucleic acids from the sample which map to one or more second genomic regions; or (ii) a mean family size distribution of sequence reads in families from the sample.

In another aspect, the present disclosure provides a method of optimising an experimental protocol, wherein the method comprises: (a) identifying whether a genomic region is subject to experimental bias using the methods disclosed herein; (b) adjusting the experimental protocol to at least partially compensate for the experimental bias identified in (a).

In some embodiments, the experimental protocol comprises hybrid capture of nucleic acids derived from target genomic regions including the genomic region identified as being subject to experimental bias, wherein hybrid capture uses probes targeted at the target genomic regions and wherein step (b) comprises adjusting the concentration of the probe to compensate for the identified experimental bias. In some embodiments, the method comprises an amplification-based enrichment using target-specific primers. In some embodiments, the parent nucleic acid molecules are amplified with universal primers followed by a second round of amplification using the target-specific primers.

In another aspect, the present disclosure provides method of predicting the experimental bias associated with a test genomic region, wherein the method comprises: (a) identifying whether a plurality of genomic regions are subject to experimental bias using the methods disclosed herein; (b) using the experimental biases identified in (a) to identify a quantitative measure of the effect of genomic factors on the experimental biases; and (c) using the quantitative measure of the effect of genomic factors on the experimental biases identified in (b) to predict the experimental bias associated with the test genomic region based on the genomic factors of the test genomic region.

In some embodiments, the genomic factors include: (i) GC content; (ii) CpG density; (iii) repetitive element frequency; (iv) epigenetic modifications, such as DNA methylation patterns or histone modifications; and/or (v) the length distribution of nucleic acid molecules mapping to the genomic region.

The methods disclosed herein can also be combined with other methods of determining a quantitative measure indicative of a number of nucleic acids in a sample, such as those methods described in PCT/US2014/072383. For example, the sequence reads in the disclosed methods may be further analyzed to determine: (i) a quantitative measure of parent DNA molecules that map to the genomic region for which both strands are detected; (ii) a quantitative measure of parent DNA molecules that map to the genomic region for which only one of the DNA strands is detected; and (iii) inferring from (i) and (ii) a quantitative measure indicative of a number of parent double-stranded DNA molecules in the sample that map to the genomic region. In some embodiments, step (iii) comprises inferring a quantitative measure of parent DNA molecules that map to the genomic region for which neither strand was detected. The grouping of sequence reads into families may be performed such that a family corresponds to sequence reads derived from the same strand of the same parent DNA molecule. Alternatively, the sequence reads derived from both strands of the same parent DNA molecule may be grouped into a single family. The quantitative measure derived from the analysis of the family size distribution and the quantitative measure derived from the analysis of the paired and unpaired strands may be combined to provide a single quantitative measure indicative of a number of DNA molecules in a sample that map to a genomic region. Alternatively, the family size distribution and the paired and unpaired strands may be analyzed in a single step to generate a single quantitative measure indicative of a number of DNA molecules in a sample that map to a genomic region.

For example, in one aspect is a method for determining a quantitative measure indicative of a number of DNA molecules in a sample that map to a genomic region, comprising: (a) providing the sample of parent DNA molecules; (b) amplifying the parent DNA molecules to provide progeny nucleic acids; (c) sequencing the progeny nucleic acids to provide sequence reads; (d) grouping the sequence reads into families, wherein a family corresponds to sequence reads derived from the same strand of a parent DNA molecule; (e) determining a quantitative measure of parent DNA molecules that map to the genomic region for which both strands are detected and a quantitative measure of parent DNA molecules that map to the genomic region for which only one of the DNA strands is detected; and (e) using: (i) the quantitative measures from step (e); and (ii) the family size distribution of families that map to the genomic region, to determine the quantitative measure indicative of the number of nucleic acids in the sample that map to the genomic region.

The various steps of the methods disclosed herein may be carried out at the same or different times, in the same or different geographical locations, e.g. countries, and/or by the same or different people.

In some embodiments, the results of the methods disclosed herein are used as an input to generate a report. The report may be in a paper or electronic format. For example, the quantitative measure indicative of a number of nucleic acids in a sample that map to a genomic region as obtained by the methods disclosed herein, or information derived therefrom, can be displayed directly in such a report. Alternatively, or additionally, diagnostic information or therapeutic recommendations which are at least in part based on the methods disclosed herein can be included in the report. In some embodiments, the results of the method or the report generated are communicated to the patient and/or healthcare provider.

The various steps of the methods disclosed herein may be carried out at the same or different times, in the same or different geographical locations, e.g. countries, and/or by the same or different people.

Additional advantages will be set forth in part in the description which follows or may be learned by practice. The advantages will be realized and attained by means of the elements and combinations particularly pointed out in the appended claims.

### DETAILED DESCRIPTION

Reference will now be made in detail to certain embodiments of the present disclosure. While the present disclosure will be described in conjunction with such embodiments, it will be understood that they are not intended to limit the invention to those embodiments. On the contrary, the present disclosure is intended to cover all alternatives, modifications, and equivalents, which may be included within the present disclosure as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids.

Numeric ranges are inclusive of the numbers defining the range. Measured and measurable values are understood to be approximate, taking into account significant digits and the error associated with the measurement.

Unless specifically noted in the above specification, embodiments in the specification that recite “comprising” various components are also contemplated as “consisting of” or “consisting essentially of” the recited components.

The section headings used herein are for organizational purposes and are not to be construed as limiting the disclosed subject matter in any way.

All patents, patent applications, websites, other publications or documents and the like cited herein whether supra or infra, are expressly incorporated by reference in their entirety for all purposes to the same extent as if each individual item were specifically and individually indicated to be so incorporated by reference. If different versions of a publication, website or the like are published at different times, the version most recently published at the effective filing date of the application is meant, unless otherwise indicated.

### Sample and Subjects

The present disclosure relates to methods of determining a quantitative measure indicative of a number of nucleic acids in a sample that map to a genomic region, e.g. DNA in a sample such as cfDNA. In some cases, the nucleic acid is obtained or has been obtained from a subject. In some embodiments, the nucleic acid sample may comprise or consist of nucleic acids, e.g. DNA, from a biological sample obtained from a subject. The subject may be a human, a mammal, an animal, a primate, rodent (including mice and rats), or other common laboratory, domestic, companion, service or agricultural animal, for example a rabbit, dog, cat, horse, cow, sheep, goat or pig. The subject may in some cases have or be suspected of having a cancer, tumor or neoplasm. In other cases, the subject may not have cancer or a detectable cancer symptom. The subject may have been treated with one or more cancer therapy, e.g., any one or more of chemotherapies, antibodies, vaccines or biologics. The subject may be in remission, e.g. from a tumor, cancer, or neoplasia (e.g., following treatment such as chemotherapy, surgical resection, radiation, or a combination thereof). The subject may or may not be diagnosed as being susceptible to cancer or any cancer-associated genetic mutations/disorders. In some embodiments, the sample is a polynucleotide sample obtained from a tumor tissue biopsy. The cancer, tumor or neoplasm may generally be of any type, for example a cancer tumor or neoplasm of the lung, colon, rectal (or colorectal), kidney, breast, prostate, or liver, or other type of cancer as described herein.

The sample can be any biological sample isolated from a subject. The sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. A sample can be isolated or obtained from a subject and transported to a site of sample analysis. The sample may be preserved and shipped at a desirable temperature, e.g., room temperature, 4° C., −20° C., or −80° C. A sample can be isolated or obtained from a subject at the site of the sample analysis.

The sample may be plasma. The volume of plasma can depend on the desired read depth for sequenced regions. Exemplary volumes are 0.4-40 ml, 5-20 ml, 10-20 ml. For examples, the volume can be 0.5 ml, 1 ml, 5 ml, 10 ml, 20 ml, 30 ml, or 40 ml. A volume of sampled plasma may be 5 to 20 ml.

A sample can comprise various amount of nucleic acid that contains genome equivalents. For example, a sample of about 30 ng DNA can contain about 10,000 (10⁴) haploid human genome equivalents and, in the case of cell free DNA (cfDNA), about 200 billion (2×10¹¹) individual polynucleotide molecules. Similarly, a sample of about 100 ng of DNA can contain about 30,000 haploid human genome equivalents and, in the case of cfDNA, about 600 billion (6×10¹¹) individual molecules.

A sample can comprise nucleic acids from different sources, e.g., cellular DNA and cell-free DNA of the same subject, or cellular DNA and cell-free DNA of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

The sample may comprise cell free nucleic acids, such as cfDNA. The cfDNA may be obtained from a test subject, for example as described above. For example, the sample for analysis may be plasma or serum containing cell-free nucleic acids. “Cell-free DNA” “cfDNA molecules,” or “cfDNA”, for example, include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) in vivo into a fluid found in the organism, and may be obtained by obtaining a sample of the fluid without the need to perform an in vitro cell lysis step. In other words, cell-free nucleic acids or cfDNA are nucleic acids or cfDNA not contained within or otherwise bound to a cell. Cell-free nucleic acids include DNA, RNA, and hybrids thereof, including cfDNA derived from genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA). In some embodiments, cell free nucleic acids are produced by tumor cells. In some embodiments, cell free nucleic acids are produced by a mixture of tumor cells and non-tumor cells.

Exemplary amounts of cell-free nucleic acids in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acids. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acids. The amount can be up to 1 femtogram (fg), 10 fg, 100 fg, 1 picogram (pg), 10 μg, 100 pg, 1 ng, 10 ng, 100 ng, 150 ng, or 200 ng of cell-free nucleic acids. The method can comprise obtaining 1 femtogram (fg) to 200 ng cell-free nucleic acids from samples.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation step in which cell-free nucleic acids, as found in solution, are separated from intact cells and other non-soluble components of the bodily fluid. Fractionation may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. Non-specific bulk carrier nucleic acids, such as C1 DNA, DNA or protein for hybridization may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Partitioning

In some embodiments of the present disclosure, the nucleic acid sample is partitioned into two or more partitions, wherein the amplification, sequencing, grouping of families and quantification are performed on at least one of the two or more partitions. In some embodiments, the sample of the parent nucleic acids has been subjected to a methylation-based partitioning assay, wherein the amplification, sequencing, grouping of families and quantification are performed on at least one of the two or more partitions of the parent nucleic acids. In some embodiments, the nucleic acid sample is partitioned based on the modification status of nucleic acids within the nucleic acid sample.

In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, the sample of parent nucleic acids are subjected to a methylation-based partitioning assay, wherein the methylation-based partitioning assay partitions nucleic acids using methyl-binding domain (MBD). In such methods, the methylation-based partitioning assay can form a hypermethylated partition and/or a hypomethylated partition. After partitioning, one or more of the resulting partitions can be analyzed by the methods disclosed herein to determine a quantitative measure indicative of a number of nucleic acids in the one or more partitions that map to a genomic region. In some embodiments, the resulting partitions analyzed can include a hypermethylated partition obtained from the methylation-based partitioning assay. In some embodiments, the resulting partitions analyzed can include a hypomethylated partition obtained from the methylation-based partitioning assay. Such methods can further comprise detecting: (i) a quantitative measure indicative of a number of nucleic acids in the hypermethylated partition; and/or (ii) a quantitative measure indicative of a number of nucleic acids in the hypomethylated partition derived from a genomic region in the sample by determining a normalized quantitative measure at one or more genomic regions and determining a methylation level at that genomic region based on the normalized quantitative measure. The methylation level can be determined, for example, by comparing the normalized quantitative measure in the hypermethylated partition to the normalized quantitative measure from the hypomethylated partition. The methylation level can be determined, for example, by comparing the normalized quantitative measure in the hypermethylated partition and/or the normalized quantitative measure from the hypomethylated partition to a reference value. The reference value may be, for example, derived from a normalized quantitative measure of a control genomic region from the same partition.

After partitioning, one or more of the resulting partitions can be analyzed by the methods disclosed herein to determine a quantitative measure indicative of a number of nucleic acids in the one or more partitions. In some embodiments, (i) the number of families that map to the genomic region; and (ii) the family size distribution of families that map to the genomic region determined through analysis of the partition can be used to infer the number of nucleic acids in a partition that map to the genomic region which did not provide any sequence reads. In some embodiments, the family size distribution of families that map to the genomic region determined through analysis of the partition can be used to determine whether the genomic region is subjected to experimental bias in a partition.

In some embodiments, the resulting partitions can include at least a hypomethylated and a hypermethylated partition obtained from the methylation-based partitioning assay. The quantitative measure determined from the sequence reads of the two or more partitions is indicative of the number of nucleic acids in the sample that map to the genomic region. The number of nucleic acids in the sample that map to a genomic region detected from the hypomethylated or hypermethylated partition would be indicative of the number of hypomethylated or hypermethylated nucleic acids in the original sample. Moreover, the determination of the number of hypomethylated or hypermethylated nucleic acids in the original sample that map to the genomic region would allow the determination of the methylation level at the genomic region.

The quantitative measure indicative of the number of hypomethylated or hypermethylated nucleic acids that map to a genomic region in the sample can be normalized to the quantitative measure at a second, or further, genomic region(s). The second, or further, genomic regions can include an internal control region of a known methylation level. The normalized quantitative measure facilitates a comparison of the number of hypermethylated and/or hypomethylated nucleic acids molecules that map to each genomic region. Hence, a methylation level of the genomic region can be determined. In some embodiments, the methylation level can be determining whether a genomic region is methylated or not methylated.

Partitioning may include physically partitioning nucleic acids into partitions based on the presence or absence of one or more methylated nucleobases. A sample may be partitioned into partitions based on a characteristic that is indicative of differential gene expression or a disease state. A sample may be partitioned based on a characteristic that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some instances, a nucleic acid sample is partitioned into two or more partitions (e g., at least 3, 4, 5, 6 or 7 partitions). The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs), including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., Methyl Minder Methylated DNA Enrichment Kit (ThermoFisher Scientific)). Subsequently, additional partitioning may involve eluting fragments having different levels of methylation by adjusting the salt concentration in a solution with the methyl binding domain and bound fragments. As salt concentration increases, fragments having greater methylation levels are eluted.

Various levels of methylation can be partitioned using sequential elutions. For example, a hypomethylated partition (no methylation) can be separated from a methylated partition by contacting the nucleic acid population with MBD, such as MBD attached to magnetic beads. The beads can be used to separate out the methylated nucleic acids from the nonmethylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation can once again be used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hypermethylated partition (enriched in nucleic acids comprising high levels of methylation). Any one or more partitions can then be analyzed using the methods disclosed herein.

In some methods, nucleic acids bound to an agent used for affinity separation-based partitioning are subjected to a wash step. The wash step washes off nucleic acids weakly bound to the affinity agent. Such nucleic acids can be enriched in nucleic acids having the modification to an extent close to the mean or median (i.e. intermediate between nucleic acids remaining bound to the solid phase and nucleic acids not binding to the solid phase on initial contacting of the sample with the agent).

For further details regarding portioning nucleic acid samples based on characteristics such as methylation, see WO2018/119452, which is incorporated herein by reference.

In some embodiments, the nucleic acids can be partitioned into different partitions based on the nucleic acids that are bound to a specific protein or a fragment thereof and those that are not bound to that specific protein or fragment thereof.

Nucleic acids can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acids based on protein bound regions. Examples of methods used to partition nucleic acids based on protein bound regions include, but are not limited to, SDS-PAGE, chromatin-immuno-precipitation (ChIP), heparin chromatography, and asymmetrical field flow fractionation (AF4).

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to: (a) MeCP2 is a protein that preferentially binds to 5-methyl-cytosine over unmodified cytosine, (b) RPL26, PRP8 and the DNA mismatch repair protein MHS6 preferentially bind to 5-hydroxymethyl-cytosine over unmodified cytosine, (c) FOXK1, FOXK2, FOXP1, FOXP4 and FOXI3 preferably bind to 5-formyl-cytosine over unmodified cytosine (Iurlaro et al., Genome Biol. 14: R119 (2013)), and (d) antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition (a residual partition) enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, the partitioned nucleic acids can be contacted with a methylation sensitive restriction enzyme (MSRE) and/or a methylation dependent restriction enzyme (MDRE). In one embodiment, a partition which is enriched for methylated nucleic acids (e.g. a hypermethylated partition and/or a residual partition) is treated with an MSRE such that unmethylated nucleic acids within the partition are digested. This can reduce the number of incorrectly partitioned nucleic acids in the partition enriched for methylated nucleic acids. In one embodiment, a partition which is unenriched for methylated nucleic acids (e.g. the hypomethylated partition) can be treated with an MDRE such that methylated nucleic acids within the partition are digested. This can reduce the number of incorrectly partitioned nucleic acids in the partition enriched for unmethylated nucleic acids.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the nucleic acids of the nucleic acid sample may be exposed to methylation-sensitive restriction enzymes (MRSEs). Such restriction enzymes do not cleave methylated residues, leaving only the methylated nucleic acids of the nucleic acid sample intact. Exposure to such restriction enzymes would result in a sample of only methylated nucleic acids, which could then be analyzed by the disclosed methods. Exposure to differential concentrations of MRSEs would result in subsamples that contain nucleic acids increasingly enriched for hypermethylated nucleic acids.

### Conversion-Based Sequencing

In some embodiments, the parent nucleic acids may be subjected to a conversion-based procedure to identify the modification status of the nucleobases. The disclosed methods can then be used to determining a quantitative measure indicative of a number of nucleic acids in a sample that map to a genomic region, including information of the nucleobase modification status of the nucleic acids. Such conversion procedures can comprise subjecting the DNA (the parent nucleic acids) to a procedure that affects a first nucleobase in the DNA differently from a second nucleobase in the DNA. In some embodiments, methods disclosed herein comprise a step of subjecting DNA, or a subsample thereof, to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA before library preparation using the DNA, before a first amplification of the DNA and/or before the ligation of adapters. In certain embodiments, the DNA is subjected to the procedure before or after contacting the DNA with a methylation-sensitive nuclease.

In some embodiments, the procedure that affects a first nucleobase in the DNA differently from a second nucleobase in the DNA comprises bisulfite conversion. Treatment with bisulfite converts unmodified cytosine and certain modified cytosine nucleotides (e.g., 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of 5-methyl cytosine (mC) and 5-hydroxymethylcytosine (hmC), such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, thus facilitates identifying positions containing mC or hmC using the sequence reads obtained from the exemplary sample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336:934-937.

In some embodiments, the procedure that affects a first nucleobase in the DNA differently from a second nucleobase in the DNA comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149:1368-80, β-glucosyl transferase can be used to protect hmC (forming 5-glucosylhydroxymethylcytosine (ghmC)), then a TET protein such as m Tet1 can be used to convert mC to caC, and then bisulfite treatment can be used to convert C and caC to U while ghmC remains unaffected. Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTetl can be used to convert mC to caC, and then bisulfite treatment can be used to convert C and caC to U while 5cmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

In some embodiments, the procedure that affects a first nucleobase in the DNA differently from a second nucleobase in the DNA comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuCL) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12 (17): e4496.

In some embodiments, the procedure that affects a first nucleobase in the DNA differently from a second nucleobase in the DNA comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv, DOI: 10.1101/2019.12.20.884692, available at www.biorxiv.org/content/10.1101/2019.12.20.884692v1. For example, TET2 and T4-BGT or 5-hydroxymethyl cytosine carbamoyltransferase (described in Yang et al., Bio-protocol, 2023; 12 (17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-BGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the procedure that affects a first nucleobase in the DNA differently from a second nucleobase in the DNA comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form P-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63:711-719. This approach is useful for identifying fragments that include one or more hmC nucleobases.

### Tagging

In some embodiments, the parent nucleic acids of the sample may be tagged with sample indexes, partition tags and/or molecular barcodes (referred to generally as “tags”). Tags can form part of an adapter.

Tags can be molecules, such as nucleic acids, containing information that indicates a feature of the molecule with which the tag is associated. For example, molecules can bear a sample tag or sample index (which distinguishes molecules in one sample from those in a different sample), a partition tag (which distinguishes molecules in one partition from those in a different partition) and/or a molecular barcode (which distinguishes different molecules from one another (in both unique and non-unique tagging scenarios). In certain embodiments, a tag can comprise one or a combination of barcodes.

Optionally, adapters may contain a partition-specific barcode and/or a molecular barcode. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally, or alternatively, for different partitions, different sets of molecular barcodes can be used such that the barcodes serve as a molecular tag through their individual sequences and also serve to identify the partition to which they correspond based the set of which they are a member.

Tags may be incorporated into or otherwise joined to adapters by chemical synthesis, ligation (e.g., as described above, e.g. by blunt-end ligation or sticky-end ligation), or overlap extension polymerase chain reaction (PCR), among other methods. Such adapters are ultimately joined to the parent nucleic acids. In other embodiments, one or more rounds of amplification cycles (e.g., PCR amplification) may be applied to introduce sample indexes to a nucleic acid using conventional nucleic acid amplification methods. The amplifications may be conducted in one or more reaction mixtures (e.g., a plurality of microwells in an array). Molecular barcodes, partition tags and/or sample indexes may be introduced simultaneously, or in any sequential order. In some embodiments, molecular barcodes and/or sample indexes are introduced prior to and/or after a partitioning procedure. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based sequence capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample nucleic acids. In some embodiments, tags are predetermined or random or semi-random sequences. In some embodiments, the tag(s) may together be less than about 500, 200, 100, 50, 20, 10, 9, 8, 7, 6, or 5 nucleotides in length. Typically, tags are about 5 to 20 or 6 to 15 nucleotides in length. The tags may be linked to sample nucleic acids randomly or non-randomly.

In some embodiments, each sample is distinctly tagged with a sample index or a combination of sample indexes. In some examples, when multiple partitions are subsequently processed after the partitioning step, each partition can be distinctly tagged with a partition tag or a combination of partition tags. In some embodiments, each nucleic acid of a sample or subsample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual nucleic acids such that the combination of the molecular barcode and the sequence of the sample nucleic acid that it is attached to creates a unique sequence that may be used for grouping the sequence reads into families, wherein a family corresponds to sequence reads derived from the same parent nucleic acid. Detection of non-unique molecular barcodes in combination with endogenous sequence information typically allows for the assignment of a unique identity to a particular molecule. Endogenous sequence information which can be used for grouping the sequence reads into families includes the beginning (start) and/or end (stop) genomic location/position corresponding to the sequence of the parent nucleic acid in the sample, start and stop genomic positions corresponding to the sequence of the parent nucleic acid in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the parent nucleic acids in the sample. In some embodiments, the beginning region comprises the first 5, the first 10, the first 15, the first 20, the first 25, the first 30 or at least the first 30 base positions at the 5′ end of the sequencing read that align to the reference sequence. In some embodiments, the end region comprises the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used for grouping the sequence reads into families, wherein a family corresponds to sequence reads derived from the same parent nucleic acid. Methylation information comprises within sequence reads, for example after bisulfite sequencing, are also optionally used for grouping the sequence reads into families, wherein a family corresponds to sequence reads derived from the same parent nucleic acid.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the parent nucleic acids) can be used. Such numbers of identifiers are typically sufficient for different molecules having the same start and stop points to have a high probability of receiving different combinations of identifiers.

In some embodiments, the assignment of unique or non-unique molecular barcodes in reactions is performed using methods and systems described in, for example, U.S. Patent Application Nos. 2001/0053519, 2003/0152490, and 2011/0160078, and U.S. Pat. Nos. 6,582,908, 7,537,898, 9,598,731, and 9,902,992. Alternatively, in some embodiments, grouping of sequence reads into families can be performed using only endogenous sequence information (e.g., start and/or stop positions, sub-sequences of one or both ends of a sequence, and/or lengths). Alternatively or additionally, in some embodiments, grouping of sequence reads into families can be performed using methylation status information, optionally in combination with other features. For example, conversion-based methylation sequencing (e.g. bisulfite sequencing) can change the base pairing specificity of bases in the parent nucleic acids depending on their methylation status, ultimately resulting in the sequence reads comprising a different nucleotide at the position of the converted base. This difference would be expected to be present all sequence reads derived from the same parent nucleic acid that had been subjected to the conversion procedure and hence can be used to group sequence reads into families. The addition of tags (e.g. sample indexes, partition and/or sub-partition tags and/or molecular barcodes) to parent nucleic acids can be done through amplification, wherein the tags are comprised in primers used for amplification.

In some embodiments, the nucleic acids are ligated to adapters comprising molecular barcodes. These molecular barcodes (optionally in combination with endogenous sequence information) can then be used for grouping the sequence reads into families, wherein a family corresponds to sequence reads derived from the same parent nucleic acid. The grouped sequence reads can then be analyzed, for example, to determine the family size distribution of families that map to a genomic region.

### Amplification

In methods of the present disclosure, parent nucleic acids within the nucleic acid sample are amplified to provide progeny nucleic acids. Amplification of the nucleic acids can be used to maximise the likelihood that the assay will detect the target sequences present within the nucleic acid sample. In some embodiments, the method includes partitioning steps wherein amplification of the nucleic acid sample can be performed before or after the partitioning steps.

Before amplification, adapters can be ligated to the sample nucleic acids, wherein the adapters comprise primer binding sites. The sample nucleic acids flanked by adapters can then be amplified by PCR and/or other amplification methods primed by primers binding to the primer binding sites in the adapters. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence-based replication.

DNA ligase can be used to ligate DNA molecules (e.g. cfDNA) in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample nucleic acid. In some instances, two adapters can be ligated to a single sample nucleic acid, with one adapter ligated to each end of the sample nucleic acid.

Ligation of adapters can comprise blunt end ligation or sticky-end ligation. In some embodiments, the present methods perform dsDNA ligations with T-tailed and C-tailed adapters when the sample nucleic acids have been subjected to A-tailing, e.g. using T4 polymerase or Klenow large fragment. This increases the efficiency of ligation and results in amplification of at least 50, 60, 70 or 80% of double stranded nucleic acids. Such methods can increase the amount or number of amplified molecules relative to control methods performed with T-tailed adapters alone by at least 10, 15 or 20%.

Adapters can include nucleic acid primer binding sites to permit amplification of a sample nucleic acid flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given nucleic acid. Adapters of the same or different sequence can be linked to the respective ends of a sample nucleic acid. In some cases, adapters of the same or different sequence are linked to the respective ends of the nucleic acid except that the sample index and/or molecular barcode differs in its sequence.

In some embodiments, primers relate to oligos which specifically target and enable amplification of amplicons within a set of amplicons. The primers may be of any suitable length depending on the particular needs and targeted sequences employed. In some embodiments, the primers may at least 10 nucleotides in length. Longer primers are also within the scope of the present disclosure as well known in the art. In some embodiments, primers may be more than 30, more than 40, more than 50 nucleotides in length.

In some embodiments, the primers used for amplification can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.). To enable hybridization to occur, primers may comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of their target sequence. In some embodiments, primers may have complete sequence identity to their target sequences.

In some embodiments, primers may contain high affinity RNA analogs such as locked nucleic acids (LNAs). LNA oligos exhibit much better thermal stability when hybridised to complementary nucleic acids compared to typical oligos. For each incorporated LNA within a primer, the melting point of the duplex increases by 2-8° C. Incorporation of LNAs into primers can be used in the disclosed methods to improve the specificity and sensitivity of the amplification reaction. In some embodiments, primers may contain molecular barcodes.

In some embodiments, amplification can comprise amplification-based enrichment. Amplification-based enrichment can comprise the use of target specific primers which specifically bind to a genomic region. The target specific primers may comprise molecular barcodes. In methods comprising amplification-based enrichment, methods may comprise: (i) amplification-based enrichment of parent nucleic acids to provide a first set of progeny nucleic acids; (ii) ligation of adapters to at least a subset of the first set of progeny nucleic acids to provide a first set of ligated progeny nucleic acids; and (iii) amplification of the first set of ligated progeny nucleic acids, using primers which bind to the adapters, to provide a second set of progeny nucleic acids. The second set of progeny nucleic acids, or derivatives thereof, may then be subjected to sequencing.

### Enrichment Using Capture Probes

Nucleic acids may be subject to a sequence capture step, in which molecules having target sequences are captured for subsequent analysis. This allows nucleic acids derived from target regions of the genome to be isolated and analyzed, thus avoiding the need for whole genome analysis. Capture can be performed before or after the amplification step.

Capture may be performed using any suitable approach known in the art. Target capture can involve use of a bait set comprising oligonucleotide baits labeled with a capture group, such as the examples noted below. The probes can have sequences selected to tile across a panel of regions, such as genes. Such bait sets are combined with a sample under conditions that allow hybridization of the target molecules with the baits. Then, captured molecules are isolated using the capture group. For example, a biotin capture group can be captured by bead-based streptavidin. Such methods are further described in, for example, U.S. Pat. No. 9,850,523.

Capture groups include, without limitation, biotin, avidin, streptavidin, a nucleic acid comprising a particular nucleotide sequence, a hapten recognized by an antibody, and magnetically attractable particles. The capture group can be a member of a binding pair, such as biotin/streptavidin or hapten/antibody. In some embodiments, a capture group that is attached to an analyte is captured by its binding pair which is attached to an isolatable moiety, such as a magnetically attractable particle or a large particle that can be sedimented through centrifugation. The capture group can be any type of molecule that allows affinity separation of nucleic acids bearing the capture group from nucleic acids lacking the capture group. An exemplary capture group are biotin which allows affinity separation by binding to streptavidin linked or linkable to a solid phase or an oligonucleotide, which allows affinity separation through binding to a complementary oligonucleotide linked or linkable to a solid phase.

In some embodiments, the methods herein comprise capturing nucleic acids comprising epigenetic and/or sequence-variable target regions. In some embodiments, the methods herein comprise capturing nucleic acids comprising epigenetic target regions, such as differentially methylated regions. Such regions may be captured from a sample (e.g., a subsample) that has undergone attachment of adapters, derivatization, partitioning, and/or amplification. Enriching for or capturing DNA comprising epigenetic and/or sequence-variable target regions may comprise contacting the DNA with a set of target-specific probes. When the method comprises a partitioning step, capturing may be performed on one or more partitions. When capturing is performed on multiple partitions, the capture probes used for each partition may be different. In some embodiments, DNA is captured from the first partition and/or the second partition and/or the unbound partition. In some embodiments, the partitions are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions. The target regions comprise intronic regions or VDJ regions that may comprise rearrangements. The target regions may comprise epigenetic target regions, which may show differences in methylation levels depending on whether they originated from a tumor or from healthy cells. The target regions may comprise sequence-variable regions, which may show differences in sequence, other than rearrangements, depending on whether they originated from a tumor or from healthy cells. The target regions may comprise both epigenetic target regions and sequence-variable regions. The capturing step produces a captured set of DNA molecules. In some embodiments, the DNA molecules corresponding to the sequence-variable target region set are captured at a greater capture yield in the captured set of DNA molecules than DNA molecules corresponding to the epigenetic target region set. In some embodiments, a method described herein comprises contacting DNA with a set of target-specific probes, wherein the set of target-specific probes is configured to capture cfDNA corresponding to the sequence-variable target region set at a greater capture yield than DNA corresponding to the epigenetic target region set. For additional discussion of capturing steps, capture yields, and related aspects, see WO2020/160414, incorporated herein by reference.

It can be beneficial to capture DNA corresponding to the sequence-variable target region set at a greater capture yield than DNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or methylation status is generally less than the volume of data needed to determine the presence or absence of genetic variants, such as cancer-related sequence mutations. Capturing the target region sets at different yields can facilitate sequencing the target regions to different depths of sequencing in the same sequencing run (e.g., using a pooled mixture and/or in the same sequencing cell).

In some embodiments, amplification is performed before the capturing step. In some embodiments, amplification is performed after the capturing step. In some embodiments, an amplification step is performed before and after the capturing step. In some embodiments, the methods further comprise sequencing the captured DNA to different degrees of sequencing depth for the epigenetic and sequence-variable target region sets and for rearrangements, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired. These can then be pooled in appropriate proportions to provide material for further processing and analysis such as sequencing.

### Sequencing

In general, sample nucleic acids flanked by adapters can be subject to sequencing after amplification. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms. Sequencing reactions can be performed in a variety of sample processing units, which may include multiple lanes, multiple channels, multiple wells, or other mean of processing multiple sample sets substantially simultaneously. Sample processing unit can also include multiple sample chambers to enable processing of multiple runs simultaneously.

Simultaneous sequencing reactions may be performed using multiplex sequencing. In some cases, cell-free nucleic acids may be sequenced with at least, for example, 1000, 2000, 3000, 4000, 5000, 6000, 7000, 8000, 9000, 10000, 50000, or 100,000 sequencing reactions. In other cases, cell-free nucleic acids may be sequenced with less than, for example, 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least, for example, 1000, 2000, 3000, 4000, 5000, 6000, 7000, 8000, 9000, 10000, 50000, or 100,000 sequencing reactions. In other cases, data analysis may be performed on less than, for example, 1000, 2000, 3000, 4000, 5000, 6000, 7000, 8000, 9000, 10000, 50000, or 100,000 sequencing reactions. An exemplary read depth is 1,000-50,000 or 1,000-10,000 or 1,000-20,000 reads per locus (base).

Sequence reads are aligned to a reference sequence, enabling the identification of reads that map to a genomic region. Prior to alignment, the sequence reads may undergo quality control analysis. Quality control analysis may perform quality control on the sequence read data from the sequencing pipeline. Only sequence reads that have passed a quality control analysis may be used by the sequence read mapper to align the sequence reads to the reference sequence. Quality control analysis of the sequence reads may include obtaining sequence reads that at least partially cover the locus of interest and analysing coverage depth based on the sequence reads that align to a reference genome above a quality threshold. The quality threshold may vary depending on the particular locus of interest involved. Examples of quality thresholds include a minimum nucleotide overlap and/or minimum alignment identity or similarity. The minimum nucleotide overlap may include, without limitation, a minimum overlap of at least about 1 base, 2 bases, 4 bases, 4 bases, 5 bases, 10 bases, 15 bases, 40 bases, 25 bases, 40 bases, 45 bases, 40 bases, 45 bases, 50 bases, 55 bases, 60 bases, 65 bases, 70 bases, 75 bases, 80 bases, 85 bases, 90 bases, 95 bases, or 100 bases. The minimum alignment identity or similarity may be at least about 50%, 55%, 60%, 65%, 70%, 75%, 80%, 85%, 90%, 95%, or more.

In some embodiments, a sequence read mapper may be used to align the sequence reads to a reference sequence. The sequence read mapper may align sequence reads using various sequence alignment technique. The reference sequence is a known sequence used for purposes of comparison with experimentally determined sequences. For example, a known sequence can be an entire genome, a chromosome, or any segment thereof. A reference can typically include at least 101, 103, 106, 109 or more nucleotides. A reference sequence can align with a single contiguous sequence of a genome or chromosome or can include non-contiguous segments aligning with different regions of a genome or chromosome. The reference sequence may include a sequence, such as a whole genome sequence, of a species of the subject. For example, if the subject is human, the reference sequence may be the hG19 or hG38 whole genome sequence. In some examples, the reference sequence may be truncated to include only the sequences of interest. For example, the hG19 or hG38 whole genome sequence may be truncated to include only the sequence corresponding to chromosome 6.

As used herein, “genomic region” refers to any region (e.g., range of base pair locations) of a genome, e.g., a gene, or an exon. A genomic region may be a contiguous or a non-contiguous region. In some embodiments, a genomic region is less than 100 Mb, less than 50 Mb, less than 20 Mb, less than 10 Mb, less than 1 Mb, less than 500 kb, less than 250 kb, less than 100 kb, less than 50 kb, less than 25 kb, less than 10 kb, less than 5 kb, less than 1 kb, less than 500 bp or less than 200 bp. In some embodiments, a genomic region is less 500 bp. In some embodiments, a genomic region corresponds to the region of the genome to which a sequence read aligns, wherein the beginning and the end of the genomic region within 10 base pairs, within 5 base pairs, within 4 base pairs, within 3 base pairs, within 2 base pairs, or within 1 base pair of the terminal alignment positions of the sequence read. In some embodiments, a genomic region corresponds to the region of the genome to which a sequence read aligns, wherein the beginning and the end of the genomic region correspond to terminal alignment positions of the sequence read.

### Family Size Distribution

In methods of the invention, the sequence reads are grouped into families, wherein the family corresponds to sequences reads derived from the same parent nucleic acid. Sequence reads from progeny nucleic acid sequences can be identified as being within the same family, therefore each family of sequences corresponds to a singular parent molecule present before amplification. Grouping the sequence reads of the progeny nucleic acids into families may be based, at least in part, on molecular barcodes contained within the sequence reads. Grouping of the sequences reads may also be based on endogenous sequence information, such as the length of the endogenous sequence reads and/or the start and/or stop position of the endogenous sequence reads when aligned to a reference sequence, as described elsewhere herein. In some embodiments, grouping the sequence reads of the progeny nucleic acids into families may be based, at least in part, on methylation information. For example, bisulfite sequencing allows the detection of methylated cytosines at a base pair resolution by converting unmethylated cytosine residues to uracil. Methylated cytosine, for example 5-methylcystosine, remains unaffected. Sequencing of the progeny nucleic acids allows for the analysis of methylation pattern at a base pair resolution. Grouping of the sequence reads into families may therefore be based, at least in part, on the methylation information comprised in the sequence reads.

In the present invention, subsequent analysis of the families, wherein a family corresponds to sequence reads derived from the same parent nucleic acids, can be used to determine a quantitative measure that is indicative of the number of nucleic acids in a sample that map to a genomic region. Specifically, the number of families that map to a specified genomic region and the distribution of the family sizes of families that map to a genomic region can be used to determine this quantitative measure. The distribution of family sizes may vary for a variety of reasons, including genomic features that introduce amplification biases for certain sequences. For instance, the GC content of the sequence, CpG density, repetitive element frequency, epigenetic modifications or the length distribution of nucleic acids molecules mapping to the genomic region.

Family size distribution refers to the statistical distribution of family sizes, wherein the family size can be measured by a variety of difference metrics. In some embodiments, the family size distribution is calculated using the counts of sequence reads within a family, wherein the number of sequence reads within a family size is used to determine the family size distribution of families that map to a genomic region. In some embodiments, the family size distribution comprises measures of central tendency of the family sizes. In some embodiments, the family size distribution comprises the mean family size of the families that map to a genomic region. In some embodiments, the family size distribution comprises the median family size of families that map to a genomic region. In some embodiments, the family size distribution comprises the mode family size of families that map to a genomic region.

In some embodiments, the family size distribution comprises the variance of family sizes of families that map to a genomic region. In some embodiments, the family size distribution comprises the standard deviation of family sizes of families that map to a genomic region. The use of metrics such as standard variation and variance to calculate family size distribution probes the spread of family sizes that map to a genomic region.

In some embodiments, the family size distribution comprises the range of family sizes of families that map to a genomic region. In some embodiments, the family size distribution comprises the interquartile range (IQR) of families that map to a genomic region. The use of the IQR as a metric for determining the family size distribution probes the spread of the family sizes, as a smaller ISR indicates that most family sizes are clustered around the median whereas a larger IQR suggests a wider spread of family sizes for a genomic region.

In some embodiments, the family size distribution comprises the proportion of families within specified size ranges that map to a genomic region. The use of proportion of families within a specified size range as a metric to calculate the distribution can provide insight into the relative frequency of different family sizes. In some embodiments, the family size distribution comprises the difference in mean family sizes between families of two specified genomic regions.

In some embodiments, the family size distribution comprises the kurtosis of family sizes of families that map to a genomic region. The kurtosis of the family sizes is a measure of the tailedness of a probability distribution. For example, in some embodiments the kurtosis of a probability distribution of mean family sizes at a specified genomic region may be used to calculate the family size distribution. Kurtosis describes how much of the probability distribution falls into the tails instead of the centre and therefor provides insight into outlier family sizes. In some embodiments, the family size distribution comprises the skewness of family sizes of families that map to a genomic region. The skewness of family sizes provides insight into whether the distribution of family sizes is symmetrical or whether the distribution is asymmetric and there is a tendency towards smaller or larger families.

### Quantification

In some aspects of the invention, the disclosure provides a method for determining a quantitative measure that is indicative of the number of nucleic acids in the sample that map to a genomic region. This quantitative measure can be determined using the number of families that map to the genomic region and the family size distribution of families that map to the genomic region.

In some embodiments, the quantitative measure can be determined by fitting the family size distribution to a statistical model. This distribution may be from the exponential distribution family. Such distributions include, but are not limited to, a Poisson distribution, negative binomial distribution, a normal distribution, a binomial distribution, or a gamma distribution. In some embodiments, the statistical model can be based on a linear model. Alternatively, the statistical model can be a non-linear model. In some embodiments, the family size distribution may be fit using a generalised-linear model wherein the outcome of the model is transformed using a link function to produce a linear relationship with the input. Generalised linear models and their respective link functions include, but are not limited to, Poisson regression using a log link function or logistic regression using a logit function.

Different algorithms and techniques can be used to fit the family size distribution to a statistical model. These algorithms and techniques are well documented in the art. Wherein the family size distribution is fit to a linear model, the least squares estimation can be used. Least squares estimation is a regression analysis based on minimizing the sum of squares of the residuals. In some embodiments, other regression estimation methods can be employed. Examples of further regression estimations for fitting the family size distribution to a statistical model include weighted least squares, penalized least squares, maximum likelihood regression, Bayesian regression, Lasso regression. Generalized linear models fit the data by identifying a set of parameters that maximize the likelihood of the data. Iterative algorithms can be used to fit the data such as iteratively re-weighted least squares (IRLS), Cyclical Coordinate Descent, Limited memory BFGS.

In some embodiments, fitting the family size distribution to a statistical model, such as a Poisson distribution or negative binomial distribution, can be carried out by training a model on the family size distribution data. An algorithm can be used to improve the fit on the statistical model. Such an algorithm can compare the processed output from the fitted model against the sample output. The correlation between these two outputs can be used to modify and improve the fit of the statistical model.

In some embodiments, the determination of the quantitative measure can comprise comparing the family size distribution to a reference value. This reference value could be the family size distribution of families of sequence reads from the sample which map to one or more second genomic regions. This reference value can also be a mean family size distribution of sequences reads in families from the sample.

Comparing the chosen reference value to the family size distribution of the genomic region can provide insight into the comparative representation of the nucleic acids in a sample that map to the genomic region, compared to nucleic acids within the sample that map to other genomic regions within the sample. Moreover, this may reveal information about bias in the genomic region. This could be indicative of experimental bias that arises due to genomic features.

For example, an increase in family size relative to a reference value may represent a bias derived from the over-representation of nucleic acids in the sample that map to the genomic region. In comparison, a decrease in family size relative to a reference value may represent a bias derived from the under-representation of nucleic acids in the sample that map to the genomic region.

In some embodiments, the analysis of the sequencing reads and the determination of a quantitative measure indicative of a number of nucleic acids in a sample that map to a genomic region can be determined using a system comprising a computer system that may include one or more computers. In some embodiments, the system, using the methods disclosed herein, is able to determine quantitative measure of cell-free nucleic acids, such as cfDNA. In some embodiments, the system may also include a sequencing system comprising one or more nucleic acid sequencers. The system may also comprise communication modules, computation modules, memory modules, and optional control modules. Note that a given module or engine may be implemented in hardware and/or in software.

Communication modules within the system may communicate frames or packets with data or information (such as measurement results or control instructions) between computers via a network (such as the Internet and/or an intranet). For example, this communication may use a wired communication protocol, such as an Institute of Electrical and Electronics Engineers (IEEE) 802.3 standard (which is sometimes referred to as ‘Ethernet’) and/or another type of wired interface. Alternatively or additionally, communication modules may communicate the data or the information using a wireless communication protocol, such as: an IEEE 802.11 standard (which is sometimes referred to as ‘Wi-Fi’, from the Wi-Fi Alliance of Austin, Texas), Bluetooth (from the Bluetooth Special Interest Group of Kirkland, Washington), a third generation or 3G communication protocol, a fourth generation or 4G communication protocol, e.g., Long Term Evolution or LTE (from the 3rd Generation Partnership Project of Sophia Antipolis, Valbonne, France), LTE Advanced (LTE-A), a fifth generation or 5G communication protocol, other present or future developed advanced cellular communication protocol, or another type of wireless interface. For example, an IEEE 802.11 standard may include one or more of: IEEE 802.11a, IEEE 802.11b, IEEE 802.11g, IEEE 802.11-2007, IEEE 802.11 n, IEEE 802.11-2012, IEEE 802.11-2016, IEEE 802.1 lac, IEEE 802.11ax, IEEE 802.11ba, IEEE 802.11be, or other present or future developed IEEE 802.11 technologies.

In some embodiments, processing a packet or a frame in a computer may include: receiving the signals with a packet or the frame; decoding/extracting the packet or the frame from the received signals to acquire the packet or the frame; and processing the packet or the frame to determine information contained in the payload of the packet or the frame. Communication may be characterized by a variety of performance metrics, such as: a data rate for successful communication (which is sometimes referred to as ‘throughput’), an error rate (such as a retry or resend rate), a mean squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). Wireless communication between components in a computer system may use one or more bands of frequencies, such as: 900 MHZ, 2.4 GHZ, 5 GHZ, 6 GHz, 60 GHz, the Citizens Broadband Radio Spectrum or CBRS (e.g., a frequency band near 3.5 GHZ), and/or a band of frequencies used by LTE or another cellular-telephone communication protocol or a data communication protocol. In some embodiments, the communication between the components may use multi-user transmission (such as orthogonal frequency division multiple access or OFDMA) and/or multiple-input multiple-output (MIMO).

Moreover, computation modules may perform calculations using: one or more microprocessors, ASICs, microcontrollers, programmable-logic devices, GPUs and/or one or more digital signal processors (DSPs). A given computation component is sometimes referred to as a ‘computation device’.

Memory modules may access stored data or information in memory that is local in a computer system and/or that is remotely located from the computer system. In some embodiments, one or more of memory modules may access stored measurement results in the local memory. Alternatively, or additionally, in other embodiments, one or more memory modules may access, via one or more of communication modules, stored measurement results in the remote memory in the computer via networks. Networks may include: the Internet and/or an intranet. In some embodiments, the measurement results are received from one or more analysis systems (such as PCR, a whole genome sequencer or a partial genome sequencer, e.g., a whole exome sequencer or, more generally, a gene sequencer that uses: a gene sequencing panel, Sanger sequencing, capillary electrophoresis and fragment analysis, microarrays, ligation-based systems, polymerase-based systems, hybridization-based systems, direct or indirect nucleotide identification systems, pyrosequencing, ion- or pH-based detection systems, electronic signature-based systems, next generation sequencing, long-read genetic sequencing, sequencing based on nanopore technology, and/or another sequencing technique) via networks and one or more of communication modules. Thus, in some embodiments at least some of the measurement results may have been received previously and may be stored in memory, while in other embodiments at least some of the measurement results may be received in real-time from the one or more analysis systems.

In some embodiments, at least a portion of the computer system is implemented at more than one location and/or by more than one person. In some embodiments, the computer system is implemented in a centralized manner, while in other embodiments at least a portion of the computer system is implemented in a distributed manner (such as using cloud-computing resources). For example, in some embodiments, the one or more analysis systems may include local hardware and/or software that performs at least some of the operations in the analysis techniques. This remote processing may reduce the amount of data that is communicated via networks. In addition, the remote processing may anonymize the measurement results that are communicated to and analyzed by the computer system. This capability may help ensure the computer system is compatible and compliant with regulations, such as the Health Insurance Portability and Accountability Act, e.g., by removing or obfuscating protected health information in the measurement results.

### Applications of Quantitative Measure

In some embodiments, the family size distribution can be used to infer the number of nucleic acids in the sample that map to a genomic region which did not provide any sequence reads. Nucleic acids in the sample that have not had their sequence read can be considered as unseen molecules. Unseen molecules in the sample can arise, for example, due to insufficient sequencing depth. However, unseen molecules can also arise from a variety of other causes including amplification errors, sequencing errors, or as a consequence of sequence length. From the determined family size distribution, the number of nucleic acids in a sample that map to a genomic region which did not provide any sequence reads can be inferred.

The process of inferring the number of nucleic acids in a sample that map to a genomic region which did not provide any sequence reads can be probabilistic. The probability that a parent nucleic acid produces no reads can be determined based on the family size distribution of a genomic region. From this probability, the number of parent nucleic acids that map to a genomic region in the original sample that did provide any sequence reads can be estimated. The probability that a parent nucleic acid produces no reads can be determined based on a statistical model that has been fit to the family size distribution of a genomic region, as described herein. Combining the measured number of “seen” and inferred number of “unseen” parent nucleic acids in the sample that map to the genomic region can provide a quantitative measure indicative of a number of nucleic acids in the sample that map to the genomic region.

In some embodiments, the quantitative measure determined by the number of families that map to the genomic region and the family size distribution of families that map to the genomic region can be normalized at one or more genomic regions. Methods of normalization are well documented in the art and provide a meaningful quantitative measure for comparison between genomic regions.

A normalized quantitative measure can be used to identify copy number variation (CNV) within a sample. CNV refers to the molecular phenomenon wherein sequences of the genome are repeated. The number of repeats vary between individuals. CNVs can have a wide range of biological implications including cancer. Significantly, CNVs have also been linked to a number of rare genomic disorders including, but not limited to, Prader-Willi syndrome and Angelman syndromes, but are also implicated in numerous common complex diseases such as neurodegenerative disorders including Parkinson's disease and Alzheimer's disease.

As described herein, a family corresponds to sequences reads derived from the same parent nucleic acid. Hence, the number of families and the size of the families can be used to infer the number of parent nucleic acids in the original sample. The normalized count of the parent nucleic acids in the original sample that map to the same genomic region can be used to estimate the copy number of the genomic region in the sample. In some embodiments, wherein the quantitative measure is determined by fitting the family size distribution to the statistical model, CNVs can be predicted from the model based on the family size distribution of the genomic region.

In some embodiments, the disclosed methods can be used to determine a tumor fraction. In some embodiments, the disclosed methods can be used to determine a mutant allele frequency (MAF). For example, the sequence reads of families can be analyzed to determine the presence or absence of mutations, such as single nucleotide variants (SNVs), insertions or deletions (indels), fusions, transversions, translocations, frame shifts, repeat variants, and epigenetic variants, such as methylation. In some embodiments, the mutations comprise SNVs. In some embodiments, the analysis of the sequence reads in a family can comprise the generation of a consensus sequence and/or the analysis of consensus base positions within sequence reads. The identification of mutations can, in turn, be used to identify the MAF for that mutation by comparing the relative frequency of families comprising the mutation and families which do not comprise the mutation. In some embodiments, the disclosed methods comprise determining: (i) a quantitative measure indicative of a number of nucleic acids in a sample that map to a genomic region which comprise a mutation; and (ii) a quantitative measure indicative of a number of nucleic acids in a sample that map to a genomic region which do not comprise a mutation. Such quantitative measures can be determined by separately analysing the number of families and family size distribution of families which do and do not comprise the mutation, respectively. In some embodiments, a tumor fraction comprises a maximum mutant allele fraction (MAF) of all somatic mutations identified in the nucleic acids.

In some embodiments, the quantitative measure is indicative of a number of nucleic acids in a hypermethylated and/or hypomethylated partition derived from a genomic region in the sample by determining a normalized quantitative measure. As described herein, in some embodiments the sample may undergo a partitioning step prior to or after amplification. This partitioning step can be a methylation-based partitioning assay wherein the assay makes use of an MBD. This partitioning assay would result in two or more partitions, including a hypomethylated partition and/or a hypermethylated partition.

In some embodiments, the quantitative measure determined from the sequence reads of the two or more partitions is indicative of the number of nucleic acids in the sample that map to the genomic region. The number of nucleic acids in the sample that map to a genomic region detected from the hypomethylated and/or hypermethylated partition would be indicative of the number of hypomethylated and/or hypermethylated nucleic acids in the original sample. Moreover, the determination of the number of hypomethylated and/or hypermethylated nucleic acids in the original sample that map to the genomic region would allow the determination of the methylation level at the genomic region.

The quantitative measure indicative of the number of hypomethylated and/or hypermethylated nucleic acids that map to a genomic region in the sample can be normalized to the same quantitative measure at a second, or further, genomic region(s). The second, or further, genomic regions can include an internal control region of a known methylation level. The normalized quantitative measure facilitates a comparison of the number of hypermethylated and/or hypomethylated nucleic acids molecules that map to each genomic region. Hence, a methylation level of the genomic region can be determined. In some embodiments, the methylation level can be determining a genomic region to be methylated or not methylated in the sample.

The disclosed methods can also reduce the sequencing depth required to accurately quantify parent nucleic acids in a sample. In more detail, the family size distribution can be used to determine a quantitative measure indicative of a number of nucleic acids in a sample that map to a genomic region, wherein the quantitative measure includes an inferred number of parent nucleic acids in the sample that map to the genomic region and that did not provide any sequence reads. This means that, even if a reduced sequencing depth is used, and thus a higher proportion of the parent nucleic acids in the sample do not provide any sequence reads, these “unseen” molecules can still be inferred and included in the quantitative measure indicative of a number of nucleic acids in a sample that map to a genomic region.

### Experimental Bias

In some embodiments, the determination of a quantitative measure includes comparing the family size distribution to a reference value, wherein the reference value can be the family size distribution of nucleic acids from the sample which map to one or more second genomic regions, or a mean family size distribution of sequence reads in the families from the sample. This comparison can inform of bias, such as experimental bias introduced in prior steps of the process, within the genomic region. For instance, an increase in family size relative to a reference value may represent a bias from the over-representation of nucleic acids in the sample that map to the genomic region. Alternatively, a decrease in family size relative to a reference value may represent a bias from under-representation of nucleic acids in the sample that map to the genomic region.

A number of genomic factors can affect bias including, but not limited to, GC content of the sequence, CpG density, repetitive element frequency within of the sequence, epigenetic modifications, such as DNA methylation patterns or histone modifications, and the length distribution of nucleic acids mapping to the genomic region. In some instances, features within the sequences can lead to differential efficiencies in amplification between different sequences. Such differences can then be exacerbated by the successive cycles of amplification.

Experimental bias can also be introduced by the experimental protocol. For example, the choice of algorithm for analysis can introduce algorithm-specific biases. The choice of algorithm during read mapping and how said algorithm deals with regions with high numbers of repeats or ambiguous matches to the reference sequence can introduce experimental biases.

In some embodiments, the family size distribution of families that map to the genomic region can be used to determine whether the genomic region is subject to bias. As previously described, this can involve the comparison of quantitative measures to reference values to identify over or under representation of specific sequences.

Identification of the experimental bias using quantitative measures, as disclosed herein, can be used to estimate a quantitative measure indicative of a number of nucleic acids in a sample that map to a genomic region in workflows wherein grouping is not performed. For example, the level of bias quantified or estimated for a particular genomic region can be used to determine an adjustment factor for that genomic region. This adjustment factor can be applied to a quantitative measure of sequence reads from that genomic region, wherein the grouping step has not been applied, to provide an adjusted quantitative measure. This adjusted quantitative measure can provide a more accurate indication the number of nucleic acids in a sample that map to a genomic region compared to if the adjustment factor had not been applied.

Identification of the experimental bias using quantitative measures, as disclosed herein, can be used to optimise the experimental protocol to reduce the observed experimental bias. Moreover, identification of the experimental bias combined with the knowledge of the sequence of the genomic region can facilitate the adjustment of specific experimental conditions to at least partially, or fully, compensate or mediate the observed experimental bias.

For example, in some embodiments, the experimental protocol comprises hybrid capture of nucleic acids derived from target genomic regions. This facilitates the enrichment of nucleic acids derived from target regions in the sample, as described elsewhere herein. In some embodiments, the nucleic acids subject to hybrid capture may include the genomic regions identified as being subject to experimental bias. The hybrid capture protocol can be adjusted to at least partially compensate for the identified bias. In further detail, hybrid capture can comprise the use of probes targeted at the target gene regions for the enrichment of the target gene regions in the samples. The concentration of the probe can be adjusted in line with the experimental bias experienced by the genomic region to allow for the at least partial compensation of the identified experimental bias. The sequence and/or length of one or more probes can be adjusted to at least partial compensate for the identified experimental bias. The design of one or more probes can be adjusted to compensate for the identified experimental bias, such as probe length, GC content, and/or complementarity to their target.

For example, in some embodiments, the experimental protocol comprises amplification-based enrichment of nucleic acids derived from target genomic regions. In some embodiments, the nucleic acids subject to amplification-based enrichment may include the genomic regions identified as being subject to experimental bias. The amplification-based enrichment protocol can be adjusted to at least partially compensate for the identified bias. In further detail, amplification-based enrichment can comprise the use of primers targeted at the target gene regions. The concentration of the primers can be adjusted in line with the identified experimental bias to allow for the at least partial compensation of the identified experimental bias. The sequence of the primers can be adjusted in line with the identified experimental bias to allow for the at least partial compensation of the identified experimental bias. For example, the primers which target under-represented nucleic acids in the sample that map to a genomic region can be redesigned to increase the GC content to make them more stable, and thus at least partially compensate for the identified experimental bias.

Compensating for such experimental biases is advantageous, for example, because it can increase the consistency of sequencing depth between genomic regions and reduces excess sequencing depth in previously over-represented genomic regions. This can lower the overall sequencing resources used per sample, thus reducing the cost of goods.

Adjusting the experimental protocol may also comprise adjusting one or more of the amplification conditions such as PCR cycle number, the polymerase used, the annealing temperature and the extension times. These factors can all affect amplification biases and as such adjustment of these factors can be used to reduce biases identified by the disclosed methods. In some embodiments, adjusting the experimental protocol may comprise adding additives to the amplification reaction to compensate for identified biases. For example, the additives may comprise betaine and/or DMSO. Betaine and DMSO reduce PCR amplification bias by stabilizing GC-rich regions and reducing secondary structure formation. Betaine lowers the melting temperature of GC-rich sequences, improving amplification efficiency, while DMSO disrupts hydrogen bonding in DNA, aiding in the amplification of difficult, high-GC, or secondary-structured templates. Accordingly, betaine and/or DMSO can be used to compensate for biases identified in GC-rich regions. In some embodiments, adjusting the experimental protocol may comprise the addition of a post-amplification normalization step, such as bead-based normalization.

### Predicting Biases Based on Genomic Factors

As previously discussed, genomic factors can affect bias. Some genomic factors that are known to introduce bias include, but are not limited to, GC content of the sequence, CpG density, repetitive element frequency within of the sequence, epigenetic modifications, such as DNA methylation patterns or histone modifications, and the length distribution of nucleic acids mapping to the genomic region. The family size distribution of families that map to a genomic region can inform and enable prediction for experimental biases in other genomic regions of interest. A quantitative measure determined by the family size distribution of families that map to a genomic region can be used in this prediction. This quantitative measure combined with the knowledge of the sequence, and therefore the genomic factors that influence bias, can be used to predict biases in other genomic regions wherein the sequence is known and the same, or different, genomic features have been identified. In other words, genomic factors in previously tested genomic regions can be correlated with a particular type and/or level of bias, and used to predict the bias of the other genomic regions (i.e. one or more test genomic regions) based on its genomic factors.

In more detail, the prediction of experimental bias associated with a test genomic region could be performed using one or more of the following steps. In some embodiments, the method involves using previously identified experimental biases to identify a quantitative measure of the effect of genomic factors on the experimental biases. This can include a step of data collection of the genomic factors of the one or more genomic regions previously analyzed. These features may include GC content, CpG density, sequence complexity, the presence of repetitive elements, secondary structure potential. Subsequently, statistical analysis can be performed to quantify the relationship between these genomic features and the observed experimental biases, such as regression analysis or machine learning techniques. This can involve creating a model where the input variables are the genomic factors, and the output variable is the degree of experimental bias (e.g., deviation in family size distribution). In some embodiments, the method comprises a step of model validation, for example using a subset of the data or cross-validation techniques to ensure its accuracy in predicting biases based on the genomic factors. The method can then be used to derive a quantitative measure from the model, such as coefficients from a regression model or feature importance scores from a machine learning model, that represents the impact of one or more genomic factors on experimental bias. This quantitative measure can then be used to predict bias in one or more test genomic regions.

The step of predicting the experimental bias associated with the test genomic region can comprise extracting the same or a subset of the genomic factors previously predicted to contribute to experimental bias (e.g., GC content, CpG density, presence of repeat sequences, potential secondary structures). These features can then be input into a predictive model, which can use the previously identified correlations to estimate the likely experimental bias for the test genomic region. For instance, if the model indicates that regions with high GC content or complex secondary structures exhibit significant underrepresentation in sequencing data, and the test genomic region shares these features, the model would predict a similar bias for the test genomic region. This prediction can then inform the design of experimental conditions, such as modifying amplification conditions and/or target enrichment conditions to at least partially compensate for the predicted bias at the test genomic region.

### Related Therapies

In certain embodiments, the methods disclosed herein relate to identifying and administering therapies, such as customized therapies, to patients or subjects based on the determination of the presence or absence or levels of epigenomic and/or genetic variation. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods.

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

In certain embodiments, the therapies can include one or more of treatments for target therapies, including abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aliqopa), crizotinib (Xalkori), dabrafenib (Tafmlar), dabrafenib mesylate (Tafmlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxetan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane 1 131 (Azedra), ipilimumab (Yervoy), isatuximab-irfc (Sarclisa), ivosidenib (Tibsovo), ixazomib citrate (Ninlaro), lanreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligeo), mosunetuzumab-axgb (Lunsumio), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride (Votrient), pembrolizumab (Keytruda), pemigatinib (Pemazyre), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), selinexor (Xpovio), selpercatinib (Retevmo), selumetinib sulfate (Koselugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-erzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), trametinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. In some embodiments, the methods comprise administering a therapy comprising a PARP inhibitor, such as olaparib, to a subject determined to have homologous recombination repair (HRR) gene or deficiency (HRD), such as with BRCA1, BRCA2, ATM, BARD1, BRIP1, CDK12, CHEK1, CHEK2, FANCL, PALB2, RAD51B, RAD51C, RAD51D, and RAD54L alterations. In some embodiments, the subject has a metastatic castrate resistant prostate cancer (mCRPC). In some embodiments, the PARP inhibitor, such as olaprib is used to treat a subject having ovarian cancer, breast cancer, pancreatic cancer, or mCRPC, wherein the subject is determined to have alterations in BRCA1, BRCA2, and/or ATM.

In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®). In certain embodiments, immunotherapy, such as pembrolizumab, is used to treat a subject determined to have a high microsatellite instability status (MSI-H). In certain embodiments, the immunotherapy, such as pembrolizumab, is used to treat a subject determined to have a high tumor mutational burden (TMB), for example, then the TMB status is greater than or equal to 10 mutations per megabase. In certain embodiment, the immunotherapy, such as pembrolizumab, is used to treat a subject determined to a have a mismatch repair deficiency (dMMR), such as in genes comprising MLH1, PMS2, MSH2 and MSH6.

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In certain embodiments, the present methods are also useful in determining the efficacy of particular treatment options. For example, the number of variations detected, irrespective of their precise identity, is a predictor of amenability to immunotherapy because the mutations create neoepitopes that can be subject of immune attack (see e.g., US20200370129).

Other variations or copy number variations indicate suitability of a particular drug. Some examples of such variations are as follows:

TABLE 1
List of cancer types with associated biomarker target and drug

| Cancer type | Drug | Target |
|---|---|---|
| Breast | abemaciclib | CDK4, CDK6 |
| Prostate | abiraterone acetate | CYP17A1 |
| Leukemia | acalabrutinib | BTK |
| Lymphoma | acalabrutinib | BTK |
| Lung | adagrasib | KRAS G12C |
| Breast | ado-trastuzumab | Tubulin, Her2 |
| | emtansine | |
| Lung | afatinib dimaleate | EGFR + EGFR exon 21 L858R mutation + |
| | | EGFR-Ex19del + HER2 + HER4 |
| Lung | alectinib | ALK, RET |
| Leukemia | alemtuzumab | CD52 |
| Skin | alitretinoin | RARα + RARβ2 + RARγ |
| Soft tissue sarcoma | alitretinoin | RARα + RARβ2 + RARγ |
| Breast | alpelisib | PI3Kα |
| Lung | amivantamab-vmjw | EGFR + c-MET |
| Breast | anastrozole | Aromatase |
| Prostate | apalutamide | AR |
| Leukemia | asciminib | Bcr-Abl |
| | hydrochloride | |
| Bladder | atezolizumab | PDL1 |
| Liver and bile duct | atezolizumab | PDL1 |
| Lung | atezolizumab | PDL1 |
| Skin | atezolizumab | PDL1 |
| Soft tissue sarcoma | atezolizumab | PDL1 |
| Gastrointestinal | avapritinib | PDGFRα + c-Kit |
| Systemic mastocytosis | avapritinib | PDGFRα + c-Kit |
| Bladder | avelumab | PDL1 |
| Endocrine and | avelumab | PDL1 |
| neuroendocrine tumors | | |
| Kidney | avelumab | PDL1 |
| Skin | Avelumab | PDL1 |
| Lymphoma | axicabtagene ciloleucel | CD19 |
| Kidney | axitinib | VEGFR1 + VEGFR2 + VEGFR3 |
| Lymphoma | belinostat | HDAC |
| Brain | belzutifan | HIF-2A |
| Kidney | belzutifan | HIF-2A |
| Pancreatic | belzutifan | HIF-2A |
| Ovarian epithelial, | bevacizumab | VEGF-A |
| fallopian tube, and | | |
| primary peritoneal | | |
| Brain | bevacizumab | VEGF-A |
| Cervical | bevacizumab | VEGF-A |
| Colorectal | bevacizumab | VEGF-A |
| Kidney | bevacizumab | VEGF-A |
| Liver and bile duct | bevacizumab | VEGF-A |
| Lung | bevacizumab | VEGF-A |
| Lymphoma | bexarotene | RXRs |
| Skin | binimetinib | MEK1, MEK2 |
| Leukemia | blinatumomab | CD19, CD3 |
| Lymphoma | Bortezomib | Proteasome |
| Multiple myeloma | bortezomib | Proteasome |
| Leukemia | bosutinib | BCR-ABL, SRC |
| Lymphoma | brentuximab vedotin | CD130, Tubulin |
| Leukemia | brexucabtagene | CD19 |
| | autoleucel | |
| Lymphoma | brexucabtagene | CD19 |
| | autoleucel | |
| Lung | brigatinib | ALK, EGFR, FLT3, IGF-1R, ROS1 |
| Prostate | cabazitaxel | Tubulin |
| Kidney | cabozantinib-s-malate | AXL + RET + ROS1 + TYRO3 + Tie-2 + |
| | | TrkB + VEGFR1 + VEGFR2 + VEGFR3 + |
| | | c-Kit + c-Met |
| Liver and bile duct | cabozantinib-s-malate | AXL + RET + ROS1 + TYRO3 + Tie-2 + |
| | | TrkB + VEGFR1 + VEGFR2 + VEGFR3 + |
| | | c-Kit + c-Met |
| Thyroid | cabozantinib-s-malate | AXL + RET + ROS1 + TYRO3 + Tie-2 + |
| | | TrkB + VEGFR1 + VEGFR2 + VEGFR3 + |
| | | c-Kit + c-Met |
| Lung | capmatinib | c-Met |
| | hydrochloride | |
| Multiple myeloma | carfilzomib | Proteasome |
| Lung | cemiplimab-rwlc | PD-1 |
| Skin | cemiplimab-rwlc | PD-1 |
| Lung | ceritinib | ALK + IGF-1R + INSR + ROS1 |
| Colorectal | cetuximab | EGFR |
| Multiple myeloma | ciltacabtagene | BCMA |
| | autoleucel | |
| Skin | cobimetinib fumarate | MEK1, MEK2 |
| Lymphoma | copanlisib | PI3Kα, PI3Kδ |
| | hydrochloride | |
| Lung | crizotinib | MTH1 |
| Lymphoma | crizotinib | MTH1 |
| Myofibroblastic | crizotinib | MTH1 |
| Brain | dabrafenib | BRAF, CRAF |
| Lung | dabrafenib mesylate | BRAF, CRAF |
| Skin | dabrafenib mesylate | BRAF, CRAF |
| Solid tumors anywhere | dabrafenib mesylate | BRAF, CRAF |
| in the body | | |
| Thyroid | dabrafenib mesylate | BRAF, CRAF |
| Lung | dacomitinib | EGFR + EGFR exon 21 L858R mutation + |
| | | EGFR-Ex19del + HER2 + HER4 |
| Multiple myeloma | daratumumab | CD38 + Hyaluronic acid |
| Multiple myeloma | daratumumab and | CD38 + Hyaluronic acid |
| | hyaluronidase-fihj | |
| Prostate | darolutamide | AR |
| Leukemia | dasatinib | Bcr-Abl + EphA2 + FYN + LCK + |
| | | PDGFRβ + Protein-tyrosine kinases + SRC + |
| | | YES1 + c-Kit |
| Neuroblastoma | dinutuximab | GD2 |
| Endometrial | dostarlimab-gxly | PD-1 |
| Solid tumors anywhere | dostarlimab-gxly | PD-1 |
| in the body | | |
| Liver and bile duct | durvalumab | PDL1 |
| Lung | durvalumab | PDL1 |
| Leukemia | duvelisib | PI3Kγ + PI3Kδ |
| Breast | elacestrant | Erα/ESR1 |
| | dihydrochloride | |
| Multiple myeloma | elotuzumab | SLAMF7 |
| Leukemia | enasidenib mesylate | IDH2 |
| Colorectal | encorafenib | BRAF, BRAF V600E |
| Skin | encorafenib | BRAF V600E, V600K mutation |
| Bladder | enfortumab vedotin- | Nectin-4, Tubulins |
| | ejfv | |
| Lung | entrectinib | ALK + ROS1 + TRKA + TrkB + TrkC |
| Solid tumors anywhere | entrectinib | ALK + ROS1 + TRKA + TrkB + TrkC |
| in the body | | |
| Prostate | enzalutamide | AR |
| Bladder | erdafitinib | FGFR1, FGFR2, FGFR3, FGFR4 |
| Lung | erlotinib hydrochloride | EGFR antagonists, EGFR exon 21 L858R |
| | | mutation inhibitors, EGFR-Ex19del |
| | | inhibitors |
| Pancreatic | erlotinib hydrochloride | EGFR + EGFR exon 21 L858R mutation + |
| | | EGFR-Ex19del |
| Breast | everolimus | mTORC1, mTORC2 |
| Kidney | everolimus | mTORC1, mTORC2 |
| Pancreatic | everolimus | mTORC1, mTORC2 |
| Breast | exemestane | Aromatase |
| Breast | fam-trastuzumab | HER2, TOP1 |
| | deruxtecan-nxki | |
| Gastric | fam-trastuzumab | HER2, TOP1 |
| | deruxtecan-nxki | |
| Lung | fam-trastuzumab | HER2, TOP1 |
| | deruxtecan-nxki | |
| Myelodysplastic and | fedratinib | JAK2, FLT3 |
| myeloproliferative | hydrochloride | |
| disorders | | |
| Breast | fulvestrant | ER |
| Liver and bile duct | futibatinib | FGFRs |
| Lung | gefitinib | EGFR + EGFR exon 21 L858R mutation + |
| | | EGFR-Exon 19 deletion |
| Leukemia | gemtuzumab | CD33, DNA |
| | ozogamicin | |
| Leukemia | gilteritinib fumarate | AXL + FLT3 |
| Leukemia | glasdegib maleate | SMO |
| Lymphoma | ibritumomab tiuxetan | CD20 |
| Leukemia | ibrutinib | BTK |
| Lymphoma | ibrutinib | BTK |
| Multiple myeloma | idecabtagene vicleucel | BCMA |
| Leukemia | idelalisib | PI3Kδ |
| Dermatofibrosarcoma | imatinib mesylate | BCR-ABL, PDGFR, C-Kit |
| protuberans | | |
| Gastrointestinal | imatinib mesylate | BCR-ABL, PDGFR, C-Kit |
| Leukemia | imatinib mesylate | BCR-ABL, PDGFR, C-Kit |
| Myelodysplastic and | imatinib mesylate | BCR-ABL, PDGFR, C-Kit |
| myeloproliferative | | |
| disorders | | |
| Systemic mastocytosis | imatinib mesylate | BCR-ABL, PDGFR, C-Kit |
| Liver and bile duct | infigratinib phosphate | FGFR1 + FGFR2 + FGFR3 + FGFR4 |
| Leukemia | inotuzumab ozogamicin | CD22 + DNA |
| Endocrine and | iobenguane I 131 | NET |
| neuroendocrine tumors | | |
| Colorectal | ipilimumab | CTLA4 |
| Esophageal | ipilimumab | CTLA4 |
| Kidney | ipilimumab | CTLA4 |
| Liver and bile duct | ipilimumab | CTLA4 |
| Lung | ipilimumab | CTLA4 |
| Malignant | ipilimumab | CTLA4 |
| mesothelioma | | |
| Skin | ipilimumab | CTLA4 |
| Multiple myeloma | isatuximab-irfc | CD38 |
| Leukemia | ivosidenib | IDH1 |
| Multiple myeloma | ixazomib citrate | proteasome |
| Endocrine and | lanreotide acetate | SSTR |
| neuroendocrine tumors | | |
| Breast | lapatinib ditosylate | EGFR |
| Solid tumors anywhere | larotrectinib sulfate | TRKA + TrkB + TrkC |
| in the body | | |
| Lung | Lazertinib | EGFR, EGFR exon 19 deletion, EGFR |
| | | exon 21 L858R substitution |
| Endometrial | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Kidney | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Liver and bile duct | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Thyroid | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Breast | letrozole/Ribociclib | CDK4/ER |
| | Succinate | |
| Lymphoma | lisocabtagene | CD19 |
| | maraleucel | |
| Lymphoma | loncastuximab tesirine- | CD19 + DNA |
| | lpyl | |
| Lung | lorlatinib | ALK + ROS1 |
| Prostate | lutetium Lu 177 | PSMA |
| | vipivotide tetraxetan | |
| Endocrine and | lutetium Lu 177- | SSTR2 |
| neuroendocrine tumors | dotatate | |
| Breast | margetuximab-cmkb | HER2 |
| Leukemia | midostaurin | FLT3 + PDGFR + PKC + Syk + |
| | | VEGFR2 + c-Kit |
| Systemic mastocytosis | midostaurin | FLT3 + PDGFR + PKC + Syk + |
| | | VEGFR2 + c-Kit |
| Ovarian epithelial, | mirvetuximab | FOLR1 + Tubulin |
| fallopian tube, and | soravtansine-gynx | |
| primary peritoneal | | |
| Lung | mobocertinib succinate | EGFR exon 20 + HER2 exon 20 |
| Lymphoma | mogamulizumab-kpkc | CCR4 |
| Lymphoma | mosunetuzumab-axgb | CD20, CD3 |
| Leukemia | moxetumomab | CD22 |
| | pasudotox-tdfk | |
| Neuroblastoma | naxitamab-gqgk | GD2 |
| Lung | necitumumab | EGFR |
| Breast | neratinib maleate | EGFR, HER2, HER4 |
| Leukemia | nilotinib | Bcr-Abl + CSF-1R + DDR1 + PDGFR + c- |
| | | Kit |
| Ovarian epithelial, | niraparib tosylate | PARP1 + PARP2 |
| fallopian tube, and | monohydrate | |
| primary peritoneal | | |
| Bladder | nivolumab | PD-1 |
| Colorectal | nivolumab | PD-1 |
| Esophageal | nivolumab | PD-1 |
| Kidney | nivolumab | PD-1 |
| Liver and bile duct | nivolumab | PD-1 |
| Lung | nivolumab | PD-1 |
| Lymphoma | nivolumab | PD-1 |
| Malignant | nivolumab | PD-1 |
| mesothelioma | | |
| Skin | nivolumab | PD-1 |
| Stomach (gastric) | nivolumab | PD-1 |
| Skin | nivolumab and | LAG3 + PD-1 |
| | relatlimab-rmbw | |
| Leukemia | obinutuzumab | CD20 |
| Lymphoma | obinutuzumab | CD20 |
| Leukemia | ofatumumab | CD20 |
| Ovarian epithelial, | olaparib | PARP1, PARP2, PARP3 |
| fallopian tube, and | | |
| primary peritoneal | | |
| Breast | olaparib | PARP1, PARP2, PARP3 |
| Pancreatic | olaparib | PARP1, PARP2, PARP3 |
| Prostate | olaparib | PARP1, PARP2, PARP3 |
| Leukemia | olutasidenib | IDH1 |
| Lung | osimertinib mesylate | EGFR + EGFR T790M + EGFR exon 21 |
| | | L858R mutation + EGFR-Ex19del |
| Myelodysplastic and | pacritinib citrate | CSF-1R + FLT3 + IRAK1 + JAK2 |
| myeloproliferative | | |
| disorders | | |
| Breast | palbociclib | CDK4, CDK6 |
| Colorectal | panitumumab | EGFR |
| Kidney | pazopanib | FGFR1 + FGFR3 + Flt3L + ITK + LCK + |
| | hydrochloride | PDGFRα + PDGFRβ + VEGFR1 + |
| | | VEGFR2 + VEGFR3 + c-Kit |
| Soft tissue sarcoma | pazopanib | FGFR1 + FGFR3 + Flt3L + ITK + LCK + |
| | hydrochloride | PDGFRα + PDGFRβ + VEGFR1 + |
| | | VEGFR2 + VEGFR3 + c-Kit |
| Breast | pembrolizumab | PD-1 |
| Cervical | pembrolizumab | PD-1 |
| Colorectal | pembrolizumab | PD-1 |
| Endometrial | pembrolizumab | PD-1 |
| Esophageal | pembrolizumab | PD-1 |
| Kidney | pembrolizumab | PD-1 |
| Liver and bile duct | pembrolizumab | PD-1 |
| Lung | pembrolizumab | PD-1 |
| Lymphoma | pembrolizumab | PD-1 |
| Skin | pembrolizumab | PD-1 |
| Solid tumors anywhere | pembrolizumab | PD-1 |
| in the body | | |
| Stomach (gastric) | pembrolizumab | PD-1 |
| Leukemia | pemigatinib | FGFR1, FGFR2, FGFR3, FGFR4 |
| Liver and bile duct | pemigatinib | FGFR1, FGFR2, FGFR3, FGFR4 |
| Lymphoma | pemigatinib | FGFR1, FGFR2, FGFR3, FGFR4 |
| Myelodysplastic and | pemigatinib | FGFR1, FGFR2, FGFR3, FGFR4 |
| myeloproliferative | | |
| disorders | | |
| Breast | pertuzumab | HER2 |
| Breast | pertuzumab, | HER2, Hyaluronic acid |
| | trastuzumab, and | |
| | hyaluronidase-zzxf | |
| Giant cell tumor | pexidartinib | CSF-1R + FLT3 + c-Kit |
| | hydrochloride | |
| Lymphoma | pirtobrutinib | BTK C481S |
| Lymphoma | polatuzumab vedotin- | CD79B + Tubulin |
| | piiq | |
| Leukemia | ponatinib hydrochloride | Bcr-Abl + Protein-tyrosine kinases |
| Lymphoma | pralatrexate | Bcr-Abl + Protein-tyrosine kinases |
| Lung | pralsetinib | RET |
| Thyroid | pralsetinib | RET |
| Prostate | radium 223 dichloride | DNA |
| Colorectal | ramucirumab | VEGFR2 |
| Gastric | ramucirumab | VEGFR2 |
| Liver and bile duct | ramucirumab | VEGFR2 |
| Lung | ramucirumab | VEGFR2 |
| Stomach (gastric) | ramucirumab | VEGFR2 |
| Colorectal | regorafenib | Abl family, BRAF, BRAF V600E, CRAF, |
| | | CSF-1R, DDR2, EphA2, FGFR1, FGFR2, |
| | | FRK, MAPK11, PDGFRa, PDGFRb, |
| | | RET, TRKA, Tie-2, VEGFR1, VEGFR2, |
| | | VEGFR3, c-kit |
| Gastrointestinal | regorafenib | Abl family, BRAF, BRAF V600E, CRAF, |
| | | CSF-1R, DDR2, EphA2, FGFR1, FGFR2, |
| | | FRK, MAPK11, PDGFRa, PDGFRb, |
| | | RET, TRKA, Tie-2, VEGFR1, VEGFR2, |
| | | VEGFR3, c-kit |
| Liver and bile duct | regorafenib | Abl family, BRAF, BRAF V600E, CRAF, |
| | | CSF-1R, DDR2, EphA2, FGFR1, FGFR2, |
| | | FRK, MAPK11, PDGFRa, PDGFRb, |
| | | RET, TRKA, Tie-2, VEGFR1, VEGFR2, |
| | | VEGFR3, c-kit |
| Skin | retifanlimab-dlwr | PD-1 |
| Breast | ribociclib | CDK4, CDK6 |
| Gastrointestinal | ripretinib | EGFR + PDGFRα + c-Kit |
| Leukemia | rituximab | CD20 + Hyaluronic acid |
| Lymphoma | rituximab | CD20 + Hyaluronic acid |
| Leukemia | rituximab and | CD20 + Hyaluronic acid |
| | hyaluronidase human | |
| Lymphoma | rituximab and | CD20 + Hyaluronic acid |
| | hyaluronidase human | |
| Lymphoma | romidepsin | HDAC |
| Ovarian epithelial, | rucaparib camsylate | PARP1 + PARP2 + PARP3 |
| fallopian tube, and | | |
| primary peritoneal | | |
| Prostate | rucaparib camsylate | PARP1 + PARP2 + PARP3 |
| Myelodysplastic and | ruxolitinib phosphate | JAK1 + JAK2 |
| myeloproliferative | | |
| disorders | | |
| Breast | sacituzumab govitecan- | TOP1 |
| | hziy | |
| Lymphoma | selinexor | XPO1 |
| Multiple myeloma | selinexor | XPO1 |
| Lung | selpercatinib | RET |
| Solid tumors anywhere | selpercatinib | RET |
| in the body | | |
| Thyroid | selpercatinib | RET |
| Plexiform neurofibroma | selumetinib sulfate | MEK1 + MEK2 |
| Soft tissue sarcoma | sirolimus protein-bound | MUT + mTOR |
| | particles | |
| Skin | sonidegib | SMO |
| Kidney | sorafenib tosylate | BRAF inhibitors, CRAF inhibitors, FLT3 + |
| | | PDGFRβ + RET + VEGFR1 + VEGFR2 + |
| | | VEGFR3 + c-Kit |
| Liver and bile duct | sorafenib tosylate | BRAF + CRAF + FLT3 + PDGFRβ + RET + |
| | | VEGFR1 + VEGFR2 + VEGFR3 + c-Kit |
| Thyroid | sorafenib tosylate | BRAF + CRAF + FLT3 + PDGFRβ + RET + |
| | | VEGFR1 + VEGFR2 + VEGFR3 + c-Kit |
| Lung | sotorasib | KRAS G12C |
| Gastrointestinal | sunitinib malate | PDGFR + RTK + VEGFR |
| Kidney | sunitinib malate | PDGFR + RTK + VEGFR |
| Pancreatic | sunitinib malate | PDGFR + RTK + VEGFR |
| Lymphoma | tafasitamab-cxix | CD19 |
| Leukemia | tagraxofusp-erzs | CD123 |
| Breast | talazoparib tosylate | PARP1, PARP2 |
| Breast | tamoxifen citrate | ER |
| Lymphoma | tazemetostat | EZH2 |
| | hydrobromide | |
| Soft tissue sarcoma | tazemetostat | EZH2 |
| | hydrobromide | |
| Skin | tebentafusp-tebn | CD3 + gp100 |
| Multiple myeloma | teclistamab-cqyv | CD3 + gp100 |
| Kidney | temsirolimus | CD3 + gp100 |
| Lung | tepotinib hydrochloride | MET |
| Leukemia | tisagenlecleucel | CD19 |
| Lymphoma | tisagenlecleucel | CD19 |
| Cervical | tisotumab vedotin-tftv | Tubulin, tissue factor |
| Kidney | tivozanib hydrochloride | (VEGFR)-1, VEGFR-2 and VEGFR-3, c- |
| | | kit, and PDGFR β |
| Breast | toremifene | ER |
| Brain | trametinib | MEK1 and MEK2 |
| Lung | trametinib dimethyl | MEK1 and MEK2 |
| | sulfoxide | |
| Skin | trametinib dimethyl | MEK1 and MEK2 |
| | sulfoxide | |
| Solid tumors anywhere | trametinib dimethyl | MEK1 and MEK2 |
| in the body | sulfoxide | |
| Thyroid | trametinib dimethyl | MEK1 and MEK2 |
| | sulfoxide | |
| Breast | trastuzumab | HER2 |
| Esophageal | trastuzumab | HER2 |
| Stomach (gastric) | trastuzumab | HER2 |
| Liver and bile duct | tremelimumab-actl | CTLA-4, CD80, and CD86 |
| Lung | tremelimumab-actl | CTLA-4, CD80, and CD86 |
| Breast | tucatinib | HER2 |
| Colorectal | tucatinib | HER2 |
| Thyroid | vandetanib | VEGFR-2, EGFR, RET |
| Skin | vemurafenib | BRAF, BRAF V600E, CRAF, ARAF, |
| | | SRMS, ACK1, MAP4K5, FGR |
| Leukemia | venetoclax | BCL-2 |
| Lymphoma | venetoclax | BCL-2 |
| Lymphoma | vorinostat | HDAC1, HDAC2 and HDAC3 (Class I) |
| | | and HDAC6 (Class II) |
| Leukemia | zanubrutinib | BTK |
| Lymphoma | zanubrutinib | BTK |
| Colorectal | ziv-aflibercept | PGF, VEGF-A |

In certain embodiments, the therapy comprises administrating a treatment to a subject determined to have a copy number amplification. In some embodiments, the treatment may comprise trastuzumab, ado-trastuzumab emtansine, or pertuzumab where the subject was determined to have an ERBB2 (HER2) gene amplification. In some embodiments, the subject has breast cancer or gastric cancer.

In some embodiments, the therapy comprises administering one or more drugs to the subject. For example, patients with non-small lung cancer determined to have either an EGFR exon 19 deletion or an EGFR exon 21 L858R alteration may be treated with amivantamab in combination with lazertinib.

The present methods can be used to generate or profile, fingerprint or set of data that is a summation of genetic information derived from different cells in a heterogeneous disease. This set of data may comprise copy number variation, nucleotide variation, epigenomic information, and/or tumor fraction. In some embodiments, the methods disclosed herein are used to monitor the efficacy or responsiveness of a treatment to the subject. In some embodiments, the methods disclosed herein can be used to determine whether the subject is a candidate for a therapy to treat the cancer or disease.

The present methods can be used to diagnose, prognose, monitor or observe cancers or other diseases of fetal origin. That is, these methodologies can be employed in a pregnant subject to diagnose, prognose, monitor or observe cancers or other diseases in an unborn subject whose DNA and other nucleic acids may co-circulate with maternal molecules.

In certain embodiments, the present methods can be used to determine minimal residual disease (MRD) of a subject, for example, based on a tumor fraction determination. In some embodiments, the methods may be directed to determining MRD by using a tissue-informed assay (i.e., using a tissue sample collected from a patient to determine a personalized panel to enrich for one or more genomic and/or epigenomic variants in a subsequent blood sample from the patient) or a tissue-naïve assay.

In certain embodiments, the present methods can integrate genomic and/or epigenomic data with proteomic (proteins and their post-translational modifications), transcriptomic, fragmentomic, immunological, histological, and/or other analyte-specific data to determine disease initiation, progression, malignant transformation, and therapeutic outcomes.

### Exemplary Embodiments

One exemplary embodiment is a method determining a quantitative measure indicative of a number of nucleic acids in a sample that map to a genomic region, comprising: (a) providing the sample of parent nucleic acids; (b) amplifying the parent nucleic acids to provide progeny nucleic acids; (c) sequencing the progeny nucleic acids to provide sequence reads; (d) grouping the sequence reads into families, wherein a family corresponds to sequence reads derived from the same parent nucleic acid; and (e) using: (i) the number of families that map to the genomic region; and (ii) the family size distribution of families that map to the genomic region, to determine the quantitative measure indicative of the number of nucleic acids in the sample that map to the genomic region.

Another exemplary embodiment is a method for determining a quantitative measure indicative of a number of nucleic acids in a hypermethylated/hypomethylated partition derived from a genomic region in the sample. The nucleic acid sample comprising the parent nucleic acids can be isolated from a plasma sample. The cfDNA sample can be incubated with a construct comprising a methylation binding domain (MBD). The MBD of the MBD construct binds to the 5mC group of the nucleic acids comprising methylated cytosines. A series of salt washes can be performed to elute progressively methylated nucleic acids to form several partitions containing nucleic acids with differing levels of methylation. Subsequently, one or more of the partitions enriched for methylated nucleic acids can be exposed to methylation-sensitive restriction enzymes (MSREs) which digest any contaminating unmethylated nucleic acids. The partitions can be provided with adapters that containing molecular barcodes. The partitions containing the parent nucleic acids undergo amplification to provide progeny nucleic acids that are sequenced and aligned to a reference sequence. The sequence reads can be grouped into families according to their parent nucleic acids using the molecular barcodes, optionally in combination with endogenous features. These families and respective family sizes can be used to determine normalized quantitative measures for one or more partitions, wherein the normalized quantitative measures are indicative of the number of nucleic acids in each partition that map to a genomic region. The number of nucleic acids in each partition can be used to determine a methylated level at the genomic region.

Another exemplary embodiment is a method for inferring from the family size distribution the number of parent nucleic acids in the sample that map to the genomic region which did not provide any sequence reads. The nucleic acid sample comprising the parent nucleic acid can be isolated from a plasma sample. During library preparation of the sample, the parent nucleic acids can be provided with adapters that contain molecular barcodes. Such barcodes can provide a method of attributing sequence reads to individual parent nucleic acids. The parent nucleic acids undergo amplification to provide progeny nucleic acids that are sequenced and aligned to a reference sequence. The sequence reads can be grouped into families according to their parent nucleic acids using the molecular barcodes, optionally in combination with endogenous features. From these families, a family size distribution of families that map to the genomic region can be determined. The family size distribution can be fit to a statistical model that is used to determine the probability that a parent nucleic acid in the original sample did not provide any sequences. This can, in turn be used to estimate the total number of parent nucleic acids in the original sample that map to a genomic region.

Another exemplary embodiment is a method for the identification of bias in a genomic region and the adjustment of the experimental protocol to at least partially compensate for the observed bias. The nucleic acid sample comprising the parent nucleic acid can be isolated from a plasma sample. During library preparation of the sample, the parent nucleic acids can be provided with adapters that contain molecular barcodes. Such barcodes can provide a method of attributing sequence reads to individual parent nucleic acids. The parent nucleic acids undergo amplification to provide progeny nucleic acids that are sequenced and aligned to a reference sequence. The sequence reads can be grouped into families according to their parent nucleic acids using the molecular barcodes, optionally in combination with endogenous features. The family size can be compared to internal control regions to determine whether the parent nucleic acid for a specific genomic region is over or under represented in the sequence reads. Using the identified experimental bias, the experimental protocol can be amended to at least partially compensate for the identified experimental bias. Wherein the experimental protocol comprises hybrid capture of nucleic acids derived from target genomic regions, including the genomic region identified as being subject to experimental bias, the concentration of probes targeted to the genomic region can be adjusted to compensate for the identified experimental bias. Moreover, the identified experimental bias can be used, combined with knowledge of the sequence of the genomic region subject to the bias, to quantitate the effect of genomic factors on the experimental biases. The effect of genomic factors can be used to predict the experimental bias in further test genomic regions based on the genomic factors in the further test genomic regions.

1. A method for determining a quantitative measure indicative of a number of nucleic acids in a sample that map to a genomic region, comprising:
(a) providing the sample of parent nucleic acids;
(b) amplifying the parent nucleic acids to provide progeny nucleic acids;
(c) sequencing the progeny nucleic acids to provide sequence reads;
(d) grouping the sequence reads into families, wherein a family corresponds to sequence reads derived from the same parent nucleic acid; and
(e) using: (i) the number of families that map to the genomic region; and (ii) the family size distribution of families that map to the genomic region, to determine the quantitative measure indicative of the number of nucleic acids in the sample that map to the genomic region.

2. The method of claim 1, wherein the method further comprises aligning the sequence reads to a reference sequence.

3. The method of claim 1, wherein the parent nucleic acids are DNA.

4. The method of claim 1, wherein the parent nucleic acids are cell-free DNA.

5. The method of claim 3, wherein the parent nucleic acids are complementary DNA (cDNA).

6. The method of claim 1, wherein step (e) comprises comparing the family size distribution to a reference value.

7. The method of claim 6, wherein the reference value is:
(i) a family size distribution of nucleic acids from the sample which map to one or more second genomic regions; or
(ii) a mean family size distribution of sequence reads in families from the sample.

8. The method of claim 1, wherein step (e) comprises inferring from the family size distribution the number of parent nucleic acids in the sample that map to the genomic region which did not provide any sequence reads.

9. The method of claim 1, further comprising detecting copy number variation in the sample by determining a normalized quantitative measure determined in step (e) at one or more genomic regions and determining copy number variation based on the normalized quantitative measure.

10. The method of claim 1, wherein the sample of parent nucleic acids has been subjected to a methylation-based partitioning assay.

11. The method of claim 10, wherein the methylation-based partitioning assay partitions nucleic acids using methyl-binding domain (MBD).

12. The method of claim 11, wherein the method is performed on: (i) a hypermethylated partition obtained from the methylation-based partitioning assay and/or (ii) a hypomethylated partition obtained from the methylation-based partitioning assay.

13. The method of claim 12, further comprising detecting a quantitative measure indicative of a number of nucleic acids in the hypermethylated and/or hypomethylated partition derived from a genomic region in the sample by determining a normalized quantitative measure determined in step (e) at one or more genomic regions and determining a methylation level at that genomic region based on the normalized quantitative measure.

14. The method of claim 1, wherein the grouping of the sequence reads into families is based at least in part on molecular barcodes.

15. The method of claim 14, wherein the molecular barcodes are attached to the parent nucleic acids through: (i) ligation of adapters comprising the molecular barcodes; or (ii) amplification using primers comprising the molecular barcodes.

16. The method of claim 1, wherein the grouping of the sequence reads into families is based at least in part on the length of the sequence reads and/or the start and/or stop position of the sequence reads when aligned to a reference sequence.

17. The method of claim 1, wherein the quantitative measure indicative of the number of nucleic acids in the sample that map to the genomic region is determined by fitting the family size distribution to a statistical model.

18. The method of claim 17, wherein the statistical model is a Poisson distribution or a negative binomial distribution.

19.-25. (canceled)
